(19) United States
(12) Patent Application Publication
(10) Pub. No.: US 2002/0078952 A1
(43) Pub. Date: Jun. 27, 2002

(54) Non-invasive sinus pain relieving assembly and method

(76) Inventor: William J. Hopper, White Salmon, WA (US)

Correspondence Address: MALLOY & MALLOY, P.A., 2800 S.W. Third Avenue, Historic Coral Way, Miami, FL, 33129, US

(21) Appl. No.: 09/992,915

(22) Filed: Nov. 16, 2001

Related U.S. Application Data
(63) Continuation-in-part of application No. 09/777,545, filed on Feb. 6, 2001.
Application No. 09/777,545 is a continuation-in-part of application No. 09/664,191, filed on Sep. 18, 2000.
Application No. 09/664,191 is a continuation of application No. 09/028,873, filed on Feb. 22, 1998, now Pat. No. 6,182,660.

(51) Int. Cl. A62B 7/00; A62B 9/00; A61M 15/00; A61M 16/00; A62B 18/00; A62B 1/00; A62B 18/02

(52) U.S. Cl. 128/200.24; 128/205.19; 128/207.13

(57) ABSTRACT

A non-invasive device and attendant method specifically designed to regulate and effectively equalize the pressure between the sinus cavities and middle ear, and the nasal cavity and nasopharynx. The relieving of the pressure within the sinus cavities in turn relieves pain normally associated with such a condition. The equalizing of pressures comprises establishing a sealing engagement about the nose and nostrils of the user and thereby effectively isolating the nasal cavity and nasopharynx, when the user establishes a palate lock, and creating a negative pressure within the nasal cavity and nasopharynx by communication with a pressure regulating assembly. Fluid in the form of mucous, liquid and/or air is thereby forced from the sinus cavities, which may define a zone of relative high pressure into relatively low pressure zone of the nasal cavity and nasopharynx, thereby providing immediate relief from sinus pain while helping to relieve middle ear pressure and associated symptoms.

### CLAIM OF PRIORITY

This is a continuation-in-part application of co-pending continuation in part application Ser. No. 09/777,545 filed on Feb. 6, 2001 which is a continuation-in-part application of presently pending U.S. patent application Ser. No. 09/664,191, which was filed on Sep. 18, 2000; which is a continuation application of U.S. patent application Ser. No. 09/028,873, which was filed on Feb. 22, 1998, which issued into U.S. Pat. No. 6,182,660 on Feb. 6, 2000. All of the above are incorporated herein by reference.

### BACKGROUND OF THE INVENTION

1. Field of the Invention

This invention relates to a non-invasive sinus pain relieving assembly and attendant methods, comprising a plurality of embodiments which are either manually or automatically activated, and structured to equalize the pressure between the sinus cavities and the middle ear by isolating the nasal cavities and nasopharynx and subjecting this area to a negative pressure. Immediate relief of normally involved sinus pain is thereby provided as well as facilitating the release of middle ear pressure.

2. Description of the Related Art

Currently, there is no commercially available, product or device available to the general public, without a prescription, that mechanically but non-intrusively relieves sinus and middle ear pressure. Present methods of treatment of sinus pain are often chemically oriented such as by the use of numerous vasoconstricting local agents or prescription drugs. Indeed, chemical dependency due to the large numbers of vasoconstrictors in use is very wide spread. In addition, tissue damage from the improper and excessive use of these vasoconstricting chemicals may lead to corrective surgery that is sometimes followed by required cosmetic surgery. Of course, surgery in the sinuses and nasal cavity can easily lead to the spread of infectious agents not only to the sensitive membranes involved but also to the bones forming these cavities.

Specifically, each sinus cavity is lined with a mucous membrane which is substantially continuous with the mucous membrane of the nose and throat. When the nose and nasal cavity fail to produce enough mucous, the sinuses are triggered to overcompensate and make up the difference. Normally, the mucous from the sinuses drain into the nasal cavity through small apertures connecting the two areas. However, if the mucous membrane of the sinuses swells or if the mucous thickens, due to at least in part to improper hydration, drainage may be impeded. If this happens, pressure inside the sinus cavities may be trapped. This could result in a differential pressure being created which physically effects, sensitive nerves and possibly often causes severe headaches or toothaches. This condition is called barotrauma. Furthermore, if outside barometric pressure increases because of changes in the weather or because of a change in altitude, such as when traveling in a descending aircraft, or auto, the condition will worsen. The same condition may effect the middle ear of a person if the bony cartilages of the eustachian tubes becomes obstructed with fluids, thereby restricting the equalizing of pressure in the middle ear. This latter condition is additionally referred to as aero otitis media.

When these conditions are encountered the sinus cavities and/or zones of the middle ear are subject to the above from numerous sources. The increase in outside pressure communicating with the nasal cavity and nasopharynx will try to communicate with the low pressure sinus cavities or middle ear, through the normally existing drain apertures, thereby reversing the flow of mucous. Upon this occurring, pressure cannot be effectively equalized by fluid transfer from a sinus and/or middle ear zone of low pressure, to the nasal cavity and nasopharynx zone of high pressure. As is well accepted, fluid will have a tendency to move from a zone of high pressure to a zone of low pressure.

A slight increase in ambient pressure on the body causes a greater pressure differential between the blood in the swollen sinus tissues and the trapped lower pressure inside the sinus cavity. Additional swelling will therefore occur in the sinus tissue. In the middle ear, the tympanic membrane or ear drum is pushed in by the higher outside pressure. This pressure differential will also cause an increase in blood flow to and a distortion of the inner ear. Sensitive nerves in the inner ear area will be effected, leading to possible pain and/or temporary hearing impairment or both. Of course the actual effects to the human body will vary depending on the size of the pressure differential as set forth above and the duration that such pressure differential is allowed to continue. However, the stress on these delicate membranes opens the door for bacterial infection that can lead to numerous well recognized complications.

Accordingly, there is a need for a non-intrusive pain relieving device which may or may not be portable and which is structured to be operable by medical personnel or, just as easily, self operated by the user of the assembly. Such an improved device and an attendant method should accomplish the rapid release of trapped pressure in the sinus cavities and middle ear, thereby serving to equalize the pressure in these zones by taking advantage of the physiological fact that all paranasal sinuses open into the lateral wall of the nasal cavity by means of small apertures to allow for the normal passage or drainage of mucous and accordingly sinus pressure. The middle ear similarly communicates with the nasopharynx by means of the eustachian tube, to drain fluids and maintain a pressure balance with the nasopharynx.

A preferred pain relieving device and method to accomplish the equalization of pressure will isolate the nasal cavity from ambient pressure conditions and establish fluid communication between a rapid acting and easily manipulated pressure regulating structure. The preferred and improved pain relieving device could comprise a plurality of different embodiments. More specifically, an improved pain relieving assembly could be manually and/or automatically operated and activated and include a pressure regulating structure which may be in the form of a mechanically expandable and contractible vacuum chamber or alternatively an electrically powered vacuum pump. Further, such an improved pain relieving assembly could be structured to activate the pressure regulating assembly, in order to create a negative pressure in the nasal cavity and nasopharynx either manually or automatically through an electrically powered drive motor. The various embodiments of the pressure regulating assembly and/or positioning assembly should be cooperatively structured to reduce the pressure in the nasal cavity and nasopharynx to a sufficient degree to force the transfer of fluid from the high pressure zones of the sinus cavities and/or middle ear to the reduced pressure zones of the nasal cavity and nasopharynx. The pressure between these zones will thereby be substantially equalized.

### SUMMARY OF THE INVENTION

The present invention relates to a portable, non-invasive sinus pain relief assembly and method for the relief of sinus pain through the equalization of pressures between the sinus cavities and the nasal cavity and/or nasopharynx. Common to the preferred embodiments of the present invention, to be described in greater detail hereinafter, is the utilization of a mask having a body portion with a hollow interior and a continuous seal structure extending about the outer periphery of the mask. The seal is specifically structured and disposed to define a sealing engagement with the face of the user in surrounding relation to the nose and nostrils. The sealing engagement is also disposed and established on a person's face so as to effectively isolate the mouth from the nose and nostrils of the user. Further, the mask, seal structure associated therewith and the establishment of the sealing engagement is such as to avoid any type of intrusive penetration of the nasal cavity through the nostrils during the placement of the mask structure and the operation of the pain relieving assembly of the present invention.

Importantly, pressure equalization of the sinus cavities in the intended manner is also dependent on isolating the nasal cavity and nasopharynx from the esophagus which is best accomplished by having the person utilizing the pain relieving assembly and practicing the attendant method creating a “palate lock”. The referred to palate lock is accomplished by the user rotating the uvula, located at the rear of the soft palate, rearwardly into engaging and sealing relation with the back of the nasopharynx. When the palate lock is established, the nasal cavity as well as the sinus cavities are thereby effectively isolated from the oral cavity, lower part of the esophagus and from exterior ambient pressure through the mouth. The nasal and sinus cavities are also isolated from external ambient pressure, due to the fact that the nostrils are maintained on the interior of the mask and within the established sealing engagement between the peripheral seal of the mask and the user's face, as set forth above. Therefore, the required isolation as well as the establishment of fluid communication between the nasal cavity and nasopharynx and the sinus cavities occurs due to the establishment of the aforementioned palate lock as well as the concurrent existence or establishment and maintenance of the sealing engagement of the seal structure so as to isolate the nose and nostrils from the mouth and ambient pressure.

At least one embodiment of the present invention comprises the provision of a pressure regulating assembly including a vacuum chamber selectively positionable between a collapsed position and an expanded position. The vacuum chamber is preferably connected in direct fluid communication to the inner end of the body portion of the mask and accordingly in direct fluid communication with the interior of the nasal cavity and nasopharynx, when the aforementioned seal of the mask is disposed in sealing engagement surrounding and effectively isolating the nasal cavity.

Further, the pressure regulating assembly of this embodiment is structured to be selectively manipulated by the hands of the user so as to cause positioning of the vacuum chamber, and parts associated therewith, between the aforementioned collapsed position and expanded position. When in the collapsed position, a minimal amount of air is present within the vacuum chamber. To the contrary, when in the expanded position, the interior volume of the vacuum chamber increases forcing a flow of air from the interior of the mask, and as a result from the interior of the nasal cavity into and/or towards the interior of the vacuum chamber. The result is an initial creation of a negative pressure within the nasal cavity and nasopharynx when the soft palate is forced into engagement with a back portion of the nasopharynx so as to establish the palate lock and thereby isolate such cavities.

Due to the natural phenomenon of the tendency of fluid to travel from a zone of high pressure towards a zone of low pressure, the initial creation of negative pressure within the nasal cavity will force the fluid flow of mucous, liquid and air through the passages normally interconnecting the sinus cavities with the nasal cavity. Similar fluid flow will result between the cavities of the middle ears and the nasopharynx. The result of course will be an equalization of pressure and the immediate relief of pain caused by swelling and by the existence of high pressure in the aforementioned middle ear and/or sinus cavities.

In accomplishing the above results, the present invention contemplates at least one preferred embodiment, wherein the pressure regulating assembly comprises a vacuum chamber which may be defined by a flexible material bellows or other structure having a hollow interior. The vacuum chamber is positionable between the aforementioned outwardly expanded position and inwardly collapsed position. The inherent flexibility and/or resiliency of the material from which the vacuum chamber or bellows is formed serves to normally bias it into the outwardly expanded position. An inwardly directed force or pressure exerted on the outer end of the vacuum chamber will cause its positioning into the collapsed position. This will vacate the majority of air within the hollow interior of the vacuum chamber.

The vacuum chamber has its inner or proximal end connected in direct fluid communication with the hollow interior of the mask and accordingly in direct fluid communication with the nasal cavity when the aforementioned peripheral seal on the mask is in sealing engagement and in surrounding relation to the nose and nostrils of the patient. As such, a release of the vacuum chamber or bellows by the user automatically forces the vacuum chamber into an outwardly expanded position due to the inherent resiliency of the bellows as set forth above. This in turn will cause a flow of air from the sealed interior of the mask and accordingly from the nasal cavity into the vacuum chamber on the interior of the bellows. An initial negative pressure will result in the interior of the nasal cavity causing any fluid (mucous, liquid and/or air) to flow from the now high pressure zone of the sinus cavities and middle ears into the now low pressure zone of the nasal cavity and nasopharynx through connecting passages normally existing in the human body. The pressure in the sinus cavities and/or middle ears will therefore be effectively equalized with the pressure in the nasal cavity and nasopharynx resulting in a relief of any pain caused by the previously existing, increased pressure in the sinus cavities and middle ears.

Yet another preferred embodiment of the present invention is defined by a somewhat similar structure as the above noted embodiment, but differs therefrom by incorporating a housing to substantially surround the bellows and allow its movement between the aforementioned expanded position and collapsed position. Further, a positioning member is attached to the outer or distal end of the bellows and manually operated by being contacted or engaged by the user, when it is desired to move the bellows from its outwardly expanded position to its inwardly collapsed position.

The housing in this embodiment further includes a lock assembly mounted at least partly thereon and partly on the positioning member, such that cooperative components of the lock assembly serve to removably retain the bellows in its collapsed position when the components of the lock assembly are engaged with one another. Accordingly the bellows, through manipulation of the positioning member, may be selectively moved into its collapsed position prior to the mask being disposed in sealing engagement about the nose and nostrils of the user. Once the bellows or vacuum chamber is retained in its collapsed position by interaction of the components of the lock assembly, the mask may be disposed in the desired sealing engagement with the user's face. When such sealing engagement is accomplished, a release assembly may be activated which serves to separate or disengage the components of the lock assembly causing the bellows to automatically move into its outwardly expanded position without further manipulation by the user. The aforementioned negative pressure is initially formed in the nasal cavity in a manner similar to that described above with reference to the other preferred embodiments of the present invention.

Yet another preferred embodiment of the structure of the present invention comprises the pressure regulating assembly being defined by a piston movably mounted within a housing, wherein the interior of the housing defines the vacuum chamber. As with the above described preferred embodiments, the piston is movable between an outwardly extending, expanded position and an inwardly disposed, collapsed position. The piston is normally biased to its outwardly expanded position by the existence of a biasing spring located within the vacuum chamber and positioned and structured to accomplish such biasing. A positioning member is movably connected to the piston and is structured to move along a specific path or track formed in the housing. When the piston is so moved, it is forced from the outwardly expanded position to the inwardly collapsed position.

Additional structural features of the positioning member and housing, relative to the travel of the piston, include an automatic release of the piston once the positioning member reaches an effective bottom or end of its inwardly directed stroke. When such occurs the piston is automatically released from its collapsed position towards and into its outwardly expanded position to accomplish the aforementioned initial negative pressure within the nasal cavity. A positioning member is then disposed in its outermost, extended position to again assume its engagement with the piston for repeated positioning of the piston into its collapsed position. Travel of the piston between the collapsed and expanded positions should occur when the mask has established the sealing engagement about the nose and nostrils of the face of the user.

In addition, a vent assembly is formed on the body and is associated with a valve structure specifically configured to allow the venting of air from the vacuum chamber as the piston travels from the outwardly expanded position to the collapsed position. The vacuum chamber is of course connected in direct fluid communication with the interior of the body of the mask so as to cause the flow of air from the nasal cavity, through the hollow interior of the mask, into the vacuum chamber, thereby establishing the initial negative pressure within the nasal cavity.

The factors set forth above which create pain within the sinus cavities can easily be compounded due to a lack of proper hydration in the nasal cavity and other affected areas. Accordingly, the present invention further contemplates an additional preferred structural embodiment and method including an attachment to the other structural components indicated above that allows steam or heated water vapor or saline solution to “pre-humidify” the nasal cavity and nasopharynx under ambient conditions. The pre-humidification occurs prior to utilization of the pain relieving assembly of the present invention and is considered a part of the method of the present invention when it is desired to pre-humidify the nasal cavity.

More specifically, a hydrator assembly comprises a housing having a hollow interior in which heated water or saline solution is placed in order to accomplish the hydration process. The upper end of the hydrator housing includes an opening and surrounding fitting which is adaptable for sealing engagement with the interior of the face mask so as to establish direct fluid communication with the interior of the hydrator housing and the interior of the face mask body. A hand manipulated valve structure is mounted on the hydrator housing and serves to selectively segregate or establish communication between the interior of the hydrator housing and the interior of the mask.

In operation, the heated water vapor from the interior of the hydrator housing rises upwardly through an opening in the top of the housing when the aforementioned valve structure is in its open position. The water vapor passes into the interior of the mask and eventually into the nasal cavity and nasopharynx. The above set forth “pre-hydration” process occurs when the face mask is disposed on the user's face an the sealing engagement is established about the nose and nostrils of the user, in segregating relation to the mouth and the pressure regulating assembly is disposed in its collapsed position.

Another preferred embodiment of the non-invasive sinus pain relieving assembly of the present invention is dimensioned and configured to preferably, but not exclusively, be portable and hand held. However, this preferred embodiment differs from the previously described embodiments in that the pressure regulating assembly comprises an electrically energized or powered vacuum pump connected in fluid communication with the mask. Further, various structural components comprising this additional preferred embodiment are mounted within a casing wherein the mask is secured to the casing and extends outwardly therefrom. As with other embodiments described herein, the mask of this embodiment includes a seal structure disposed substantially about the periphery of an open outer end thereof. The seal may be formed from a conforming gel or other material which facilitates the establishment of a sealing engagement with the users face. In particular, the sealing engagement is formed in surrounding relation to the nose and nostrils of the user so as to isolate the nose and nostrils from the mouth. As set forth above, by virtue of existence of the sealing engagement between the seal structure and the user's face, the nasal cavity and nasopharynx are isolated from ambient pressure and are exteriorly isolated from the users mouth or oral cavity.

Other structural features of this preferred embodiment include a source of electrical energy, such as a removable and/or rechargeable battery assembly, mounted within the casing and electrically connected to the vacuum pump. Further, a switch assembly, preferably in the form of an on/off switch is connected in current regulating relation between the source of electrical energy and the vacuum pump. The vacuum pump is connected in direct fluid communication with the hollow interior portion of the mask and thereby serves to create a predetermined negative pressure within the nasal cavity and nasopharynx when the seal structure is disposed in the aforementioned sealing engagement about the nose and nostrils.

In use, a mask, vacuum pump or pressure regulating assembly, as well as the other components of this embodiment of the present invention are disposed in an “operative position”. The referred to operative position is at least partially defined by the establishment of the seal structure of the mask in the sealing engagement with the user's face, as set forth above. The user then establishes a “palate lock” immediately prior to or concurrently with the activation of the vacuum pump. The palate lock, as set forth above, is accomplished when the user positions the uvula rearwardly into engaging and sealing relation to the back of the nasopharynx. The establishment of the palate lock by the user serves to effectively isolate the nasal cavity as well as the sinus cavities from the oral cavity and the lower part of the esophagus as well as from exterior ambient pressures, as the mouth of the patient is closed during the establishment of the palate lock. As a further result, the sinus cavities are also isolated from external ambient pressure due to the fact that the nostrils and nose are disposed within the mask and are surrounded and isolated by the establishment of the sealing engagement of the seal structure.

Further, when in the operative position and upon activation of the vacuum pump, a negative pressure is established within the nasal cavity and nasopharynx, which is now isolated, in the manner set forth above. The negative pressure is sufficient to facilitate the flow of fluid (mucus, liquid, air, etc.) from the sinus cavities, now at a relatively higher pressure, to the lower pressure zones of the nasal cavity and nasopharynx.

Another important feature of this preferred embodiment of the present invention comprises the provision of regulating assembly preferably including a pressure relief assembly. An inner end of the mask is connected in sealing engagement with the casing and in direct fluid communication with the vacuum pump so as to generate the aforementioned negative pressure within the mask, nasal cavity and nasopharynx of the user. A connecting seal or O-ring type of seal structure serves to sealing connect the inner end of the mask to the casing to facilitate the establishment and transfer of the negative pressure into the interior of the mask.

However, the pressure relief assembly is associated with the connecting seal of the mask, at least to the extent of being able to cause the partial displacement thereof. Such displacement creates an at least minimal communication between the interior of the mask and the exterior of the mask or casing. Accordingly, upon an indication of excessive pressure and/or upon the negative pressure within the mask reaching a predetermined level, the pressure relief assembly will be automatically actuated so as to at least partially displace the connecting seal and thereby “open” the sealing attachment between the mask and the casing. The interior of the mask will thereby be at least minimally exposed to ambient, external pressure thereby forcing the negative pressure within the mask to fall into a preferred and/or predetermined range.

As will be described in greater detail hereinafter, the pressure relief assembly may also be operable to eliminate the negative pressure within the mask upon completion the treatment. Also, the pressure relief assembly may automatically perform a “cycling operation” such that the negative pressure created by the vacuum pump and communicated to the interior of the mask is continuously maintained within the predetermined pressure range in order to efficiently effect the desired treatment as described herein.

The sinus pain relieving assembly of the present invention comprises yet another preferred embodiment which utilizes the same principal of isolating the nasal cavity and nasopharynx so as to establish a negative pressure therein. Moreover, equalization of pressure between the sinus cavities and the nasal cavity thereby occurs, as with the embodiments described above, even though the operational and structural features of this additional preferred embodiment differs.

More specifically, this preferred embodiment of the present invention comprises a housing including a pressure regulating assembly mounted therein in the form of a vacuum chamber. The vacuum chamber may or may not comprise a bellows or like flexible and/or resilient material chamber positionable between an expanded position and a collapsed position. In the expanded position the volume of the interior chamber is increased and in the collapsed position the volume of the interior chamber is significantly decreased or voided. The vacuum chamber of the pressure regulating assembly is connected in fluid communication with a mask which, as in the other embodiments described herein, has a peripheral seal mounted thereon. The peripheral seal and mask is dimensioned and configured to be disposed in overlying, surrounding relation to the nose and nostrils of the user and is disposed and structured to form a sealing engagement with the face of the user. The sealing engagement is more specifically defined by a substantially fluid tight or fluid flow restricting seal which isolates the nose and nostrils from ambient pressures as well as the mouth of the user.

One distinguishing feature of this preferred embodiment comprises a positioning assembly including an automatic drive structure which serves to drivingly engage and position the vacuum chamber into both the expanded position and the collapsed position. The automatic drive structure of the positioning assembly is preferably defined by a drive motor which is preferably electrically powered or energized. The drive motor is connected in driving relation to a positioning member in the form of a carriage which is disposed in orienting or positioning engagement with the vacuum chamber to facilitate its movement between the aforementioned collapsed and expanded positions.

As with the pressure regulating assembly of certain of the above noted embodiments, the vacuum chamber defined herein is structured to create a negative pressure as the vacuum chamber passes from the collapsed position into the expanded position due to a continuous increase in the interior volume thereof. Due to the fact that the interior of the vacuum chamber is disposed in fluid communication with the interior of the mask, air will be drawn from the mask into the interior of the vacuum chamber. Further, once the sealing engagement is established between the peripheral seal of the mask and the face of the user, the created negative pressure will be transferred to the nasal cavity and nasopharynx of the user, thereby significantly lowering the pressure therein.

Further, the pressure regulating assembly will be specifically designed and structured to create a sufficient amount of negative pressure to establish fluid transfer between the sinus cavities and the nasal cavity. Due to the natural tendency of fluid to flow from a zone of high pressure to a zone of lower pressure, trapped mucous, liquid, air, etc. within the sinus cavities will flow through normally existing connecting passages into the nasal cavity. A substantial equalization of the pressure between the sinus cavities and the nasal cavity as well as the zones of the middle ear will thereby occur. Pressure will be released, fluid will be transferred and the result will be a significant an immediate relief of pain originating in the sinus cavities and/or zones of the middle ear as set forth in greater detail above.

In this preferred embodiment of the pain relieving assembly of the present invention, the pressure regulating assembly associated therewith comprises not only the vacuum chamber but also a safety member which are connected to one another and which are replaceable as a single unit. Therefore, a mounting structure is attached to the housing of the pain reliever and is cooperatively structured with a connector or attachment member associated with the pressure regulator assembly. As such, the pressure regulating assembly is intended to be removed after a single use in order to avoid contamination between consecutive patients or users of this embodiment of the present invention.

Accordingly, after each use, the pressure regulating assembly is detached from the mask and the housing is opened to facilitate removal of a previously used pressure regulating assembly. An insertion of a new, unused pressure regulating assembly may occur at the same time. In addition, the safety member associated with each of the plurality of replaceable pressure regulating assemblies is disposed and structured to structurally “modify” the vacuum chamber in a manner which renders it unuseable. As such, the safety member may be in the form of a lance or other piercing device which penetrates a wall portion of the vacuum chamber once the vacuum chamber is disposed in an extreme collapsed position.

In order to facilitate the automatic positioning of the vacuum chamber between the aforementioned collapsed and expanded positions, as well as forcing the vacuum chamber into an extreme collapsed position, during which it is penetrated by the safety member, the positioning assembly activates the pressure regulating assembly through both a load cycle and a treatment cycle, as described in more detail hereinafter. During the required load cycle of operation the previously used vacuum chamber is penetrated or otherwise structurally modified so that it can not be re-used. The treatment cycles serves to activate or operate the vacuum chamber through the collapsed and expanded position in order to create the sufficient negative pressure to accomplish equalization between the sinus cavities and the nasal cavity as set forth above.

### BRIEF DESCRIPTION OF THE DRAWINGS

For a fuller understanding of the nature of the present invention, reference should be had to the following detailed description taken in connection with the accompanying drawings in which:

FIG. 1 is a schematic view in partial section indicating the nasal cavity, nasopharynx and sinus cavity and their effective pressurization during operation of the subject invention.

FIG. 2 is a side view in partial perspective showing one preferred embodiment of the present invention in an operative position on a user.

FIG. 3 is a sectional view of the assembled components of the embodiment of FIG. 2.

FIG. 4 is a sectional view in detached and exploded form of the embodiment of FIG. 2.

FIG. 5 is side view in partial section of yet another preferred embodiment of the present invention in an operative position on the face of the user.

FIG. 6 is a detailed sectional view of the embodiment of FIG. 5.

FIG. 6A is a detailed sectional view of a variation of the embodiment of FIG. 5 including external perimeter threading to achieve detachment of the mask.

FIG. 7 is a detailed sectional view of certain structural components of the embodiment of FIGS. 5 and 6 where a pressure regulating assembly thereof is shown in an expanded position.

FIG. 8 is yet another preferred embodiment of the present invention.

FIG. 9 is a sectional view in partial cutaway of the embodiment of FIG. 8 in a different operative position.

FIG. 10 is a sectional view in partial cutaway of the embodiment of FIGS. 8 and 9 in a different operative position.

FIG. 11 is a sectional view in partial cutaway of the embodiment of FIGS. 8, 9 and 10 in a different operative position.

FIG. 12 is a sectional view in partial cutaway of the embodiments of FIGS. 8-11 in a different operative position.

FIG. 13 is a perspective view of yet another preferred embodiment of the present invention relation to a hydrator assembly.

FIG. 14 is a sectional view in partial cutaway showing the hydrator assembly of FIG. 13 attached to the structural embodiment of FIG. 6.

FIG. 15 is a top perspective view of another preferred embodiment of the pain relieving assembly of the present invention shown in an assembled view.

FIG. 16 is a front view of the embodiment of FIG. 15.

FIG. 17 is a perspective view of the embodiment of FIGS. 15 and 16.

FIG. 18 is a rear perspective view of the embodiment of FIGS. 15-17.

FIG. 19 is a perspective view of the operative and structural components absent a surrounding casing of the embodiment of FIGS. 15 and 16.

FIG. 20 is a different perspective view of the embodiment of FIG. 17.

FIG. 21 is a rear perspective view of the embodiment of FIGS. 17 and 18.

FIG. 22 is an interior sectional view of another preferred embodiment of the pain relieving assembly of the present invention.

FIG. 23 is a sectional view in partial phantom and cutaway of a mask associated with the preferred embodiment of FIG. 22 and attached to a portion thereof.

FIG. 24 is a sectional view in partial cutaway showing an operative position of a positioning assembly comprising a part of the preferred embodiment of FIG. 22 in a different operative position from FIG. 23.

FIG. 25 is a transverse sectional view of a pressure regulating assembly along line 25-25 of FIG. 23.

FIG. 26 is an interior sectional view of the embodiment of FIG. 1 in a different operative position.

FIG. 27 is a partially exploded, interior sectional view of the preferred embodiment of FIG. 22 disclosing a replaceable component thereof.

Like reference numerals refer to like parts throughout the several views of the drawings.

### DETAILED DESCRIPTION OF THE PREFERRED EMBODIMENT

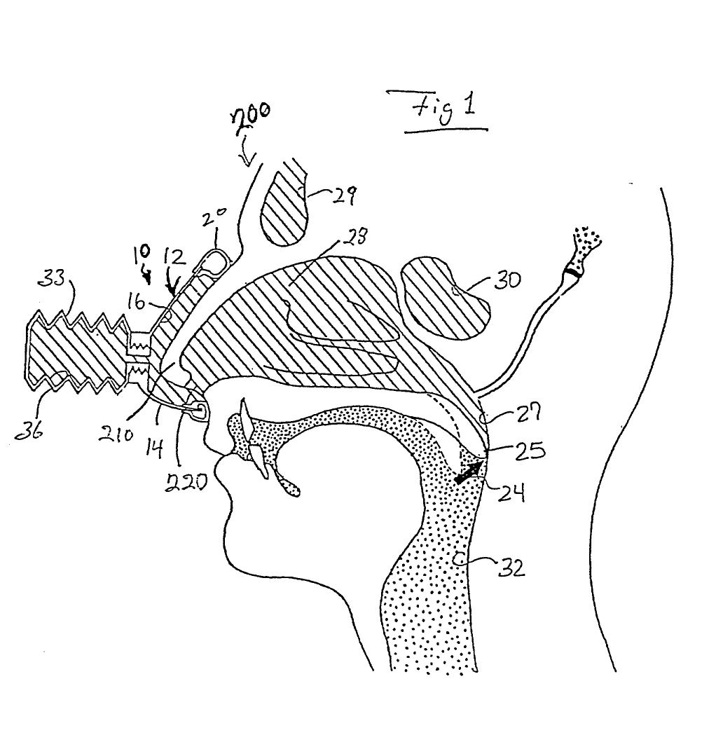

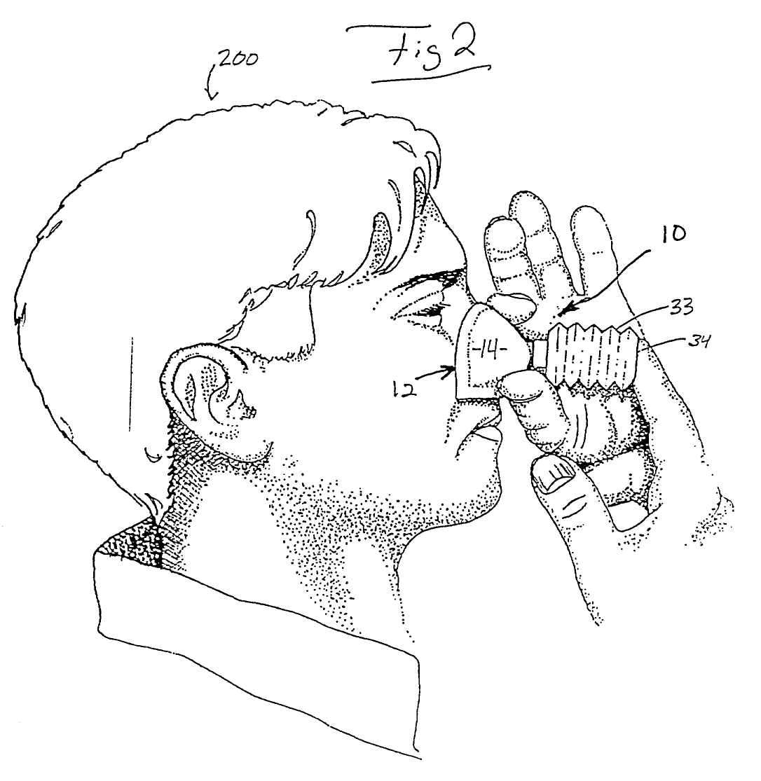

The present invention is directed towards a non-invasive sinus pain relief assembly having one preferred embodiment thereof generally indicated as 10 and shown in an operative position in FIGS. 1 and 2. More specifically, with reference to FIG. 1 the various effective cavities of the patient or user of the subject assembly 10 are delineated for purposes of explanation. The assembly 10 includes a preferably small, mask, generally indicated as 12, having a body portion 14 with a hollow interior as at 16. The outer end of the body portion 14 includes a continuous peripheral seal structure 20 structured to establish a sealing engagement on the face of the user 200. More specifically, a substantially fluid tight seal is created in surrounding relation to the nose 210 and the nostrils 220. An important feature of the present invention is the structuring of the preferred embodiments herein so as to be non-invasive in that the method of the present invention and the operation of the subject assembly 10, as well as the other preferred embodiments is practiced without entering the nasal cavity per se, through the nostrils 220. Moreover, while any preferably resilient material seal 20 can be employed to achieve mating, generally air-tight engagement with the face and nose of a user, in one preferred embodiment the seal 20 is formed from a self conforming material such as a hydro-plastic material or gel. Specifically, the entire mask 12 or merely the seal portion thereof can be at least partially structured of this self forming material, such that a user can conform the seal 20 to the contours of their face, and can achieve a sealing engagement which sufficiently restricts fluid flow between the seal 20 and the user's face to allow for a predetermined negative pressure to be maintained within the mask 12. Further, the forming material comprising at least a portion of the seal 20 can be shaped either by placing the mask 12 on the users face, or with some materials, by heating or otherwise treating the material to permit the forming or shaping. Such an embodiment is particularly suited when the device is to be used by many individuals, with each individual having their own detachable mask 12.

Again with reference to FIG. 1 the establishment of a “palate lock” is disclosed by the directional arrow 24, indicating a rearward rotation of the uvula 25, located at the end of the soft palate, into engaging and substantially sealing engagement with the back of the nasopharynx generally as at 27. The establishing of the palate lock serves to effectively isolate the nasal cavity 28, as well as the indicated sinus cavities 29 and 30, from the lower part of the esophagus 32 and from exterior ambient pressures through the oral cavity and mouth, when the mouth is closed. This isolation occurs due to the existence of the sealing engagement of the peripheral seal 20 in surrounding and spaced relation to the nose and nostrils 210 and 220 of the user 200.

As will be explained in greater detail hereinafter, the sinus pain relief assembly in the embodiments of FIGS. 1-4 includes a pressure regulating assembly in the form of bellows 33. The bellows 33 are formed from a flexible plastic or like material which is inherently biased to assume the outwardly expanded position as shown in FIGS. 1, 2 and 3, and may include a resilient material bulb or a piston structure. A preferably threaded connector structure as at 35 serves to removably connect the inner or proximal end of the bellows 33 at the inner end of the body portion 14 and in direct fluid communication with the hollow interior 16 of the body portion 14 of mask 12.

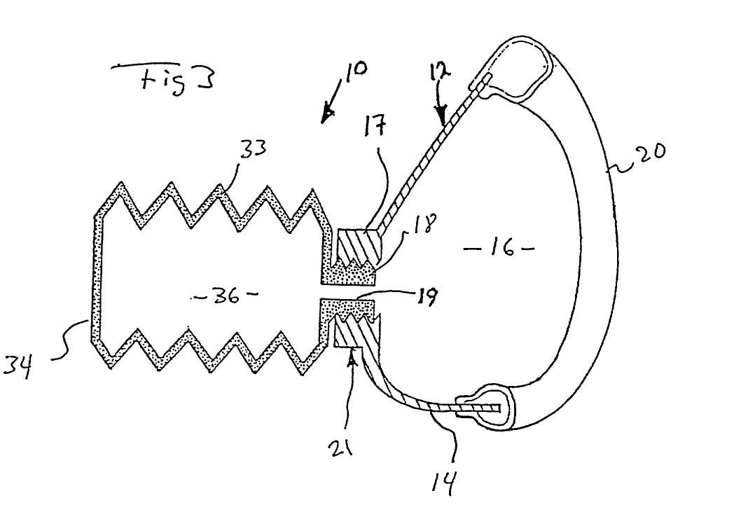

The placing of an inwardly directed pressure by the user 200, such as by utilizing their finger, (See FIG. 2) on the outer or distal end 34 of the bellows 33 will cause it to move from the outer, expanded position, where a maximum amount of air is contained within the vacuum chamber 36 defined within the interior of the bellows 33, to an inwardly directed collapsed position, wherein a minimal amount of air is contained within the vacuum chamber 36. Preferably, this collapsed position is achieved before the assembly 10 is sealed against the user's face. When, however, the vacuum chamber 36 is positioned in its collapsed position and once the seal 20 is in sealing engagement about the nose and nostrils of the user 100, the patient facilitates isolation of the lower esophagus 32 by swallowing. Swallowing causes the rearward rotation, in accordance with the directional arrow 24, of the end of the soft palate 25 into engagement with the nasopharynx 27. Indeed, once sealed against a user so as to enclose the nose, there is a low pressure state inside the sinus cavities and nasopharynx, and there is insufficient air movement to move liquids into the assembly 10. As the bellows moves to the expanded position shown in FIGS. 1 and 3, a flow of air, which was under negative tension, will be caused to travel from the interior of the nasal cavity 28 into the hollow interior 16 of the body portion 14 and eventually into the interior of the vacuum chamber 36 of bellows 33. This will initially cause a negative pressure within the nasal cavity 28 and nasopharynx 27. Due to normally existing passages and apertures existing between the sinus cavities 29 and 30 and the nasal cavity 28, the initial negative pressure within the nasal cavity 28 will cause a flow of fluid (mucous, liquid, air) from what may be a now existing high pressure zone in the sinus cavities 29 and 30 into the now low pressure zone of the nasal cavity 28 through the aforementioned interconnecting passages and apertures. The pressure will thereby be equalized between the nasal cavity 28 and the sinus cavities 29 and 30. For purposes of disclosure, certain linear cross hatching existing in FIG. 1 represents the initial negative pressure being exerted on the nasal cavity 28 and effectively on the sinus cavities 29 and 30 as the vacuum chamber 36 and bellows expand from its collapsed position to the pictured, expanded position shown in FIGS. 1 and 3. After use by an individual, the assembly 10 can be removed from its operative position by the user 200 “releasing” the palate lock as by simply swallowing completing a swallow.

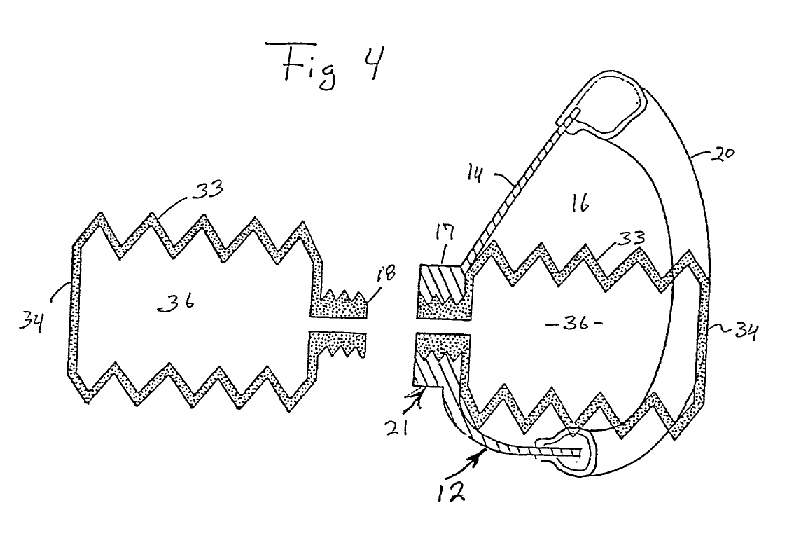

With reference to FIGS. 3 and 4, the preferred embodiment of the present invention further comprises a connector 21, preferably defined by a female, internally threaded collar 17 integrally formed on one end of the body portion 14 and specifically structured to be removably connected to an externally threaded male member 18 preferably integrally formed on the inner or proximal end of the bellows 33. A passage as at 19 is formed within the male member 18 so as to establish direct fluid communication between the hollow interior 16 of the body portion 14 and the vacuum chamber 36 defined on the interior of the bellows 33.

With reference to FIG. 4, the connector 21 is preferably removably connected so as to allow detachment of the male member 18 from the female collar 17 and from its operative position into a stored position wherein the bellows 33 is removably secured and disposed in the hollow interior 16 of the body portion 14 as shown.

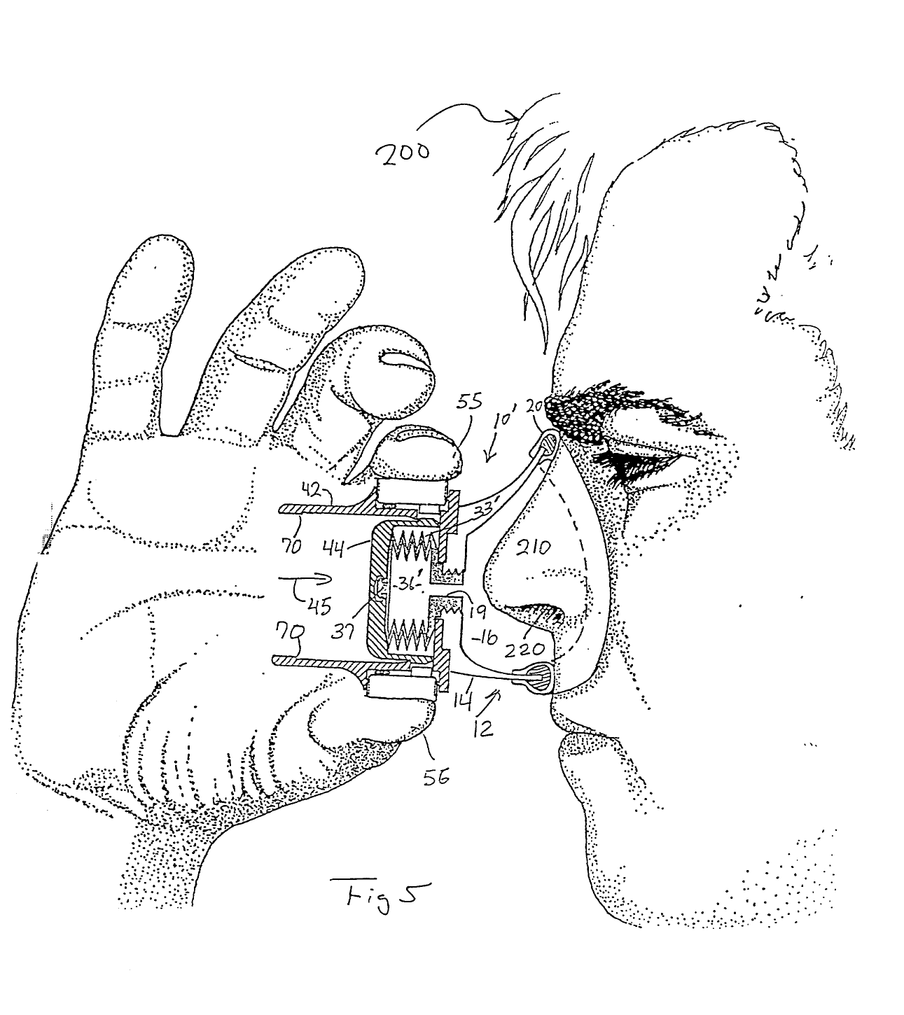

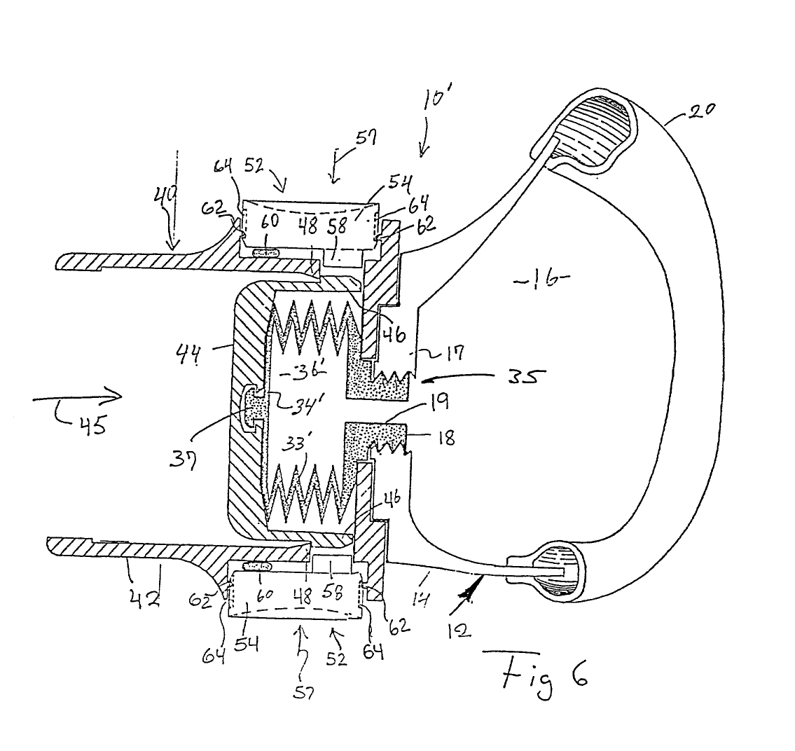

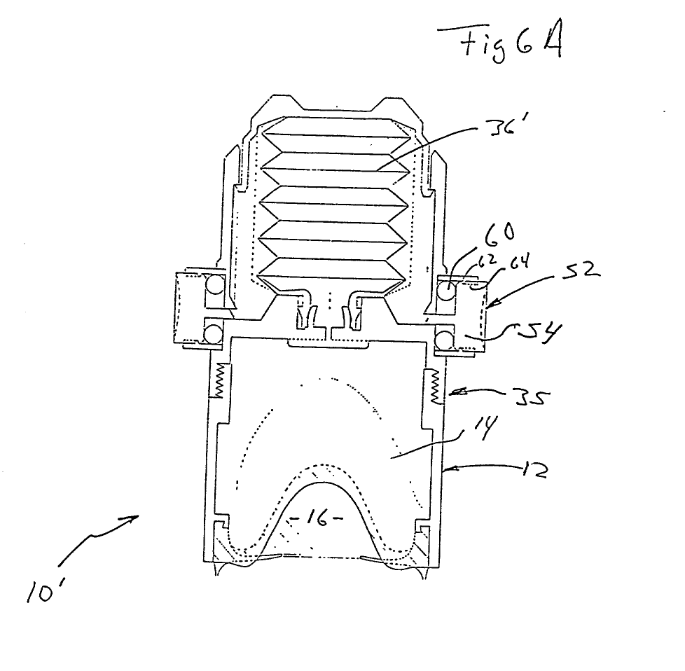

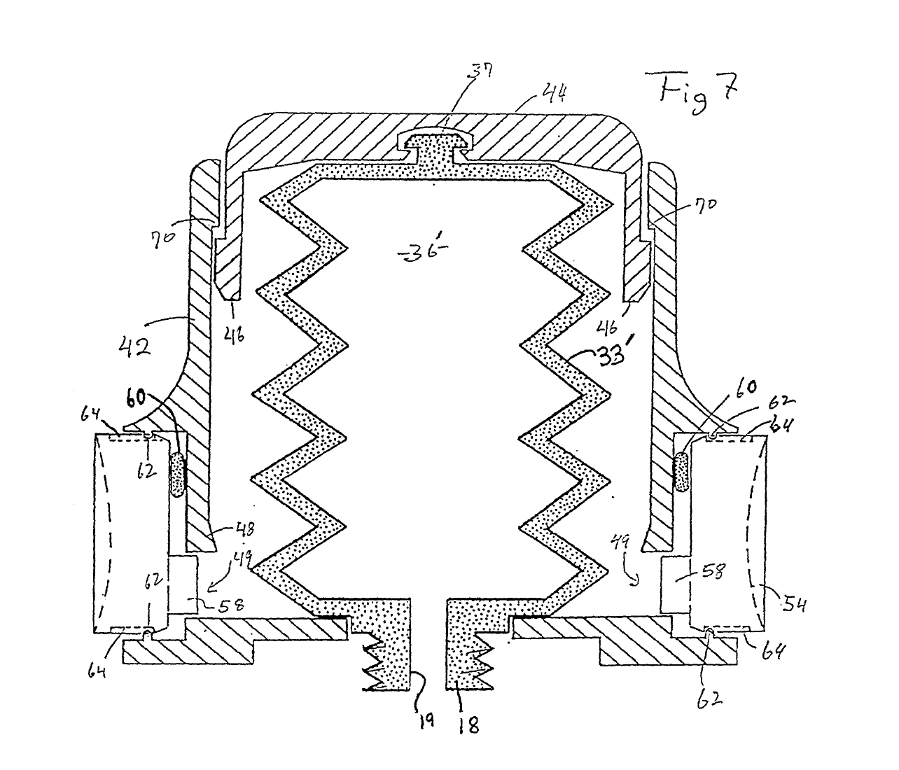

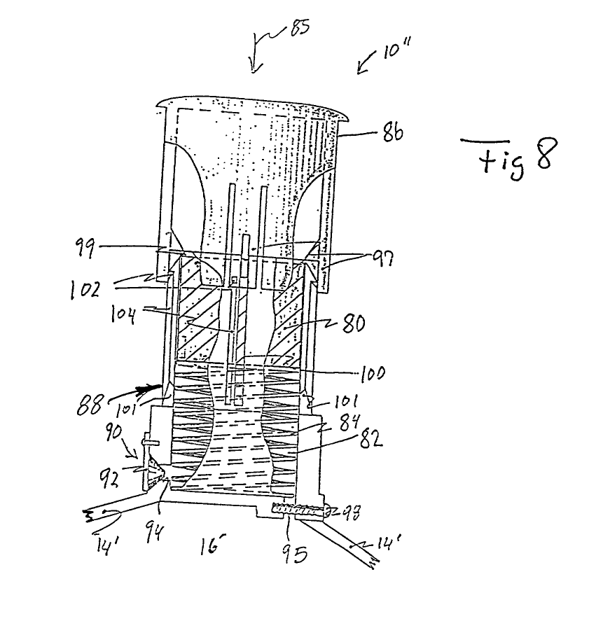

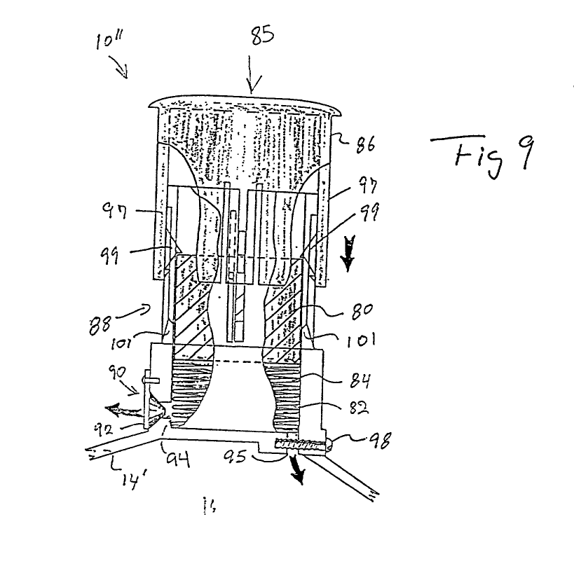

Looking to FIGS. 5, 6 and 7, the present invention comprises yet another preferred embodiment of the sinus pain relief assembly, generally indicated therein as 10′. This embodiment of the sinus pain relief assembly 10′ is similar to that of the previously recited preferred embodiment sinus pain relief assembly 10 in that the pressure regulating assembly is primarily defined by an expandable bellows 33′ formed of a plastic or like flexible material which is normally biased outwardly into the expanded position shown in FIG. 7. The vacuum chamber 36′ is defined on the hollow interior of the bellows 33′ and the bellows 33′ is movable selectively from the expanded position of FIG. 7 to the collapsed position of FIG. 6 so as to remove the majority of air from the vacuum chamber 36′. Once so removed, the seal 20 is engaged in sealing relation and surrounding disposition about the nose 210 and an outwardly spaced relation to the nostrils 220 of the user 200.

Additional structural features of the preferred embodiment sinus pain relief assembly 10′ include the existence of a housing generally indicated as 40 and including a somewhat sleeve like structure 42 disposed in surrounding, somewhat enclosing relation to the bellows 33′ when it is either in its expanded position of FIG. 7 or its collapsed position of FIG. 6. Further, the sinus pain relief assembly 10′ includes a positioning member 44 secured to the outer or distal end as at 34′ of the bellows 33′. For example, although not necessary as in FIG. 6A, an integrally formed projection as at 37 of FIG. 5 may be used if desired. The positioning member 44 is connected to the bellows 33′ so as to travel therewith and accomplish the selective positioning of the bellows 33′ from its expanded position of FIG. 7 to its collapsed position of FIG. 5 and 6. Such disposition is accomplished similarly to that of FIG. 10 in that a single finger of the user may be positioned on the exterior of the positioning member 44 and an inwardly directed force is applied thereto in accordance with directional arrow 45.

Also preferably included with the sinus pain relief assembly 10′ of the present invention is a locking assembly. The locking assembly comprises one or more first locking components 46 integrally formed on the positioning member 44 and movable therewith. A second locking component comprises one or more inwardly directed shoulders as at 48 inwardly extending into the path of travel of the first locking components 46 of the positioning member 44. With reference to FIG. 6, inwardly directed force applied to the positioning member 44 will serve to position it until the second locking members 48 removably but lockingly engage the first locking component 46 on the positioning member as shown. It should be apparent that the bellows 33′ is therefore retained within the collapsed position. The seal 20 is then applied in sealing engagement with the face and in surrounding relation to the nose 210 and nostrils 220 of the user 200. When properly sealed and when the hollow interior 16 of the body portion 14 and the vacuum chamber 36′ are in direct fluid communication with one another a release mechanism generally indicated as 52 is activated. Such activation occurs by an inwardly directed force being exerted on release members 54 by appropriately positioned fingers 55 and 56 of the user 200. Such inwardly directed force in accordance with directional arrows 57 will cause fingers 58 attached to the members 54 to engage the first locking components 46 on the positioning member 44. This will cause an inward flexing of the first locking members 46 and a disengagement of these locking members 46 from the second locking members 48. The inherent flexibility and structural bias of the bellows 33′ will thereby cause the bellows to move outwardly into the expanded position shown in FIG. 7. A negative pressure will be created in the nasal cavity 28 (See FIG. 1) as air flows therefrom through the hollow interior 16 of the body portion 14 and through passage 19 into the interior of the vacuum chamber 36′. Operation of the release assembly 52 is facilitated through the provision of biasing members as at 60 serving to normally position the release members 54 in there outermost position as shown in FIG. 6. Projections 62 preferably integrally formed on the housing body 42 slide within the tracks or grooves integrally formed in the release member 54 so as to properly orient and position the release member 54 for operation as described above.

With reference to FIG. 7, one or more block members as at 70 are integrally formed on the housing body 42 and limit the outermost extension of the positioning member 44 through engagement with the first locking components 46 as clearly disclosed.

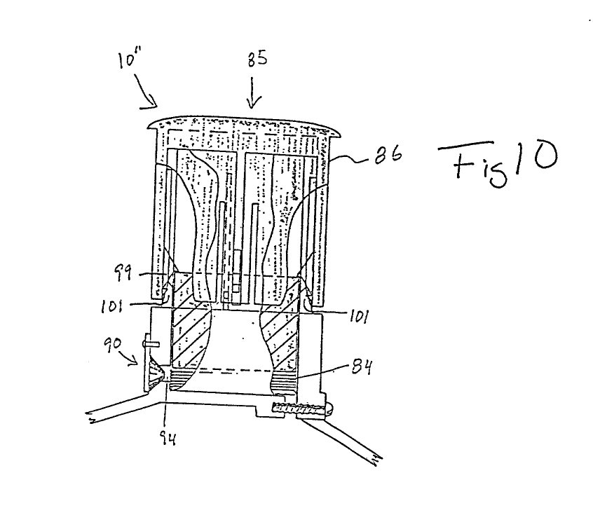

Yet another preferred embodiment of the sinus pain relief assembly 10″ is shown in various operative positions in FIGS. 8-12. More specifically, the sinus pain relieving assembly 10″ includes a movable vacuum piston 80 movably mounted within a vacuum chamber 82 and biased by a biasing spring 84 into its normally outer, expanded position, as shown in FIG. 8. The movement of the piston 80 is accomplished by manipulation of a positioning member 86 initially connected to an outermost end of the piston 80, as best shown in FIG. 8. The pressure regulating assembly includes a body 88 having a hollow interior which defines the vacuum chamber 82 and in which the piston 80 moves between the aforementioned expanded positions of FIG. 8 and the collapsed position of FIG. 11. A vent assembly 90 is formed on the housing 88 of the pressure regulating assembly and comprises a valve member 92 preferably in the form of a flap valve serving to normally seal a vent aperture 94. However, when the piston 80 is directed inwardly into the vacuum chamber 82 and into its collapsed position (See FIG. 11). Air normally within the vacuum chamber 82 will be exhausted through vent aperture 94 as the flap valve 92 is forced outwardly to release air pressure therein. The positioning member 86 is of course manipulated by an appropriate finger of the hand of the user to apply the force as indicated by directional arrow 85. Similarly, a vacuum passage as at 95 serves to establish direct fluid communication between the vacuum chamber 82 and the hollow interior 16′ of the body portion 14′ as well as the interior of the nasal cavity 28 once the seal 20 (not shown) is in its aforementioned sealed engagement on the face of the user. An adjusting screw is disposed and structured to regulate the size of vacuum passage 95.

Other structural features of the additional embodiment of the sinus pain relief assembly 10″ includes a lock and unlock components in the form of elongated arms as at 97 being formed of a flexible material and integrally attached to the positioning member 86. In addition, piston engagement tangs as at 99 are also integrally formed on the lock and unlocking arms 97 so as to travel therewith. In the position shown in FIG. 8 these tangs 99 engage the outermost end of the piston 80 and force it downwardly from the expanded position of FIG. 8 into the collapsed position of FIG. 11. Such tangs 99 travel in piston engaging tang slots 100 formed in the housing 88 of the pressure regulating assembly for guidance of the positioning member 85 during the reciprocal travel during its stroke. Further, an unlocking shoulder as at 101 is also included and integrally formed on the housing 88 and disposed in interruptive relation to the downward travel of the piston engaging tangs 99. Once the shoulders 101 engage the tangs 99 the lock and unlocking arms 97 will be forced outwardly (See FIG. 11) causing the release of the positioning member 86 from the piston and an upward travel of the positioning member as well as the beginning upward travel of the piston 80. Upward travel into its expanded position of the piston 80 will be caused by the force exerted thereon will by the biasing spring 84.

An up lock 102 formed on the positioning member 86 is structured and disposed to travel within up lock grooves 104 formed on the housing 88 and serve to limit the outer extension of the positioning member 86 as best shown in FIG. 8. A thorough review of the various operative positions shown in FIGS. 8-12 show that a downward force 85 exerted on the positioning member 86 will cause a downward travel of the piston 80 into its collapsed position as shown in FIG. 10. The continued downward travel of the positioning member 86 will in turn cause an engagement of the unlocking shoulders 101 with the tangs 99 of the locking and unlocking arms 97. The positioning member 86 will therefor be disengaged from the piston allowing the upward traveling of the piston 80 due to the force exerted thereon by the biasing spring 84 and the eventual upward travel of the positioning member 86.

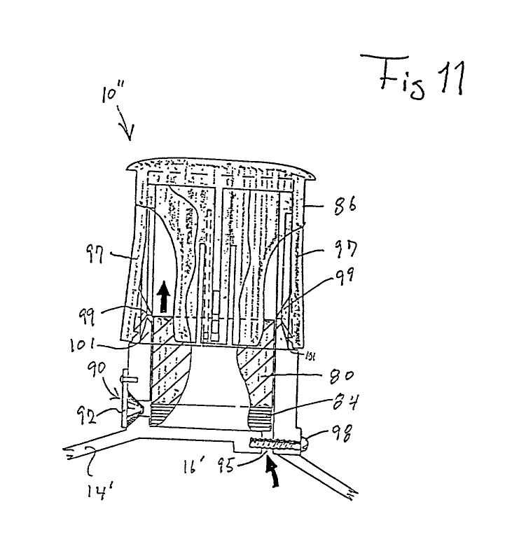

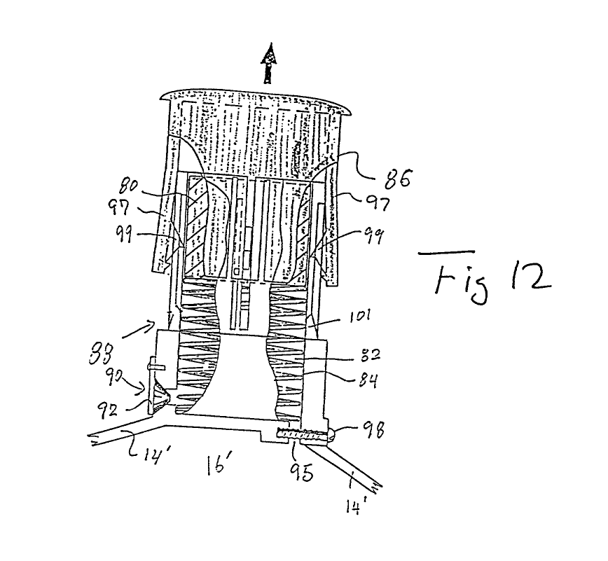

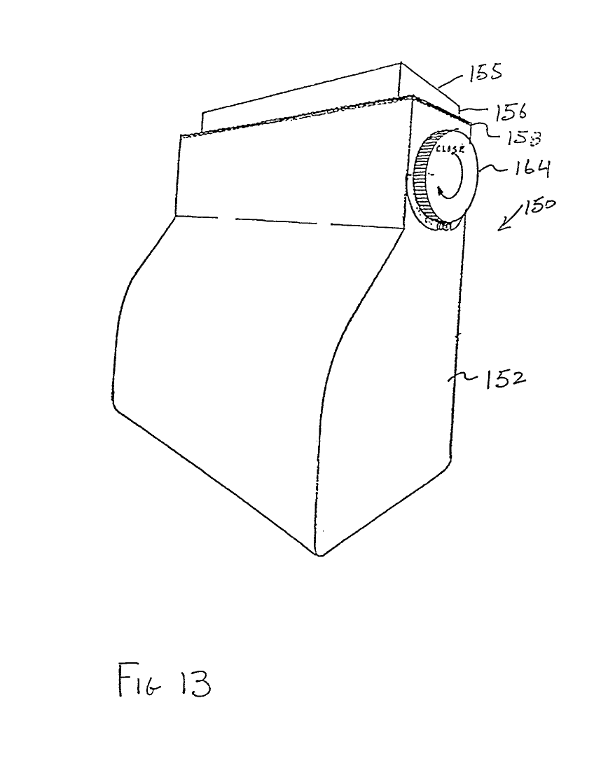

Yet another preferred embodiment of the present invention is shown in FIGS. 13 and 14 wherein a hydrator assembly generally indicated as 150 includes a hydrator housing 152 having a hollow interior (See FIG. 14) 153. The upper end of the housing 152 has an opening as at 155 surrounded by a mating or connecting collar 156. A gasket or seal structure 158 is disposed in surrounding relation to the connecting collar 156 and is specifically adapted to engage an outer surface of the body portion 14′ of the mask in order to establish sealing engagement therewith. The upper opening or aperture 155 communicates directly with the hollow interior 16 of the body portion 14′ in that the connecting or mating collar 156 passes through a specifically dimensioned and configured opening as at 160 integrally formed in the body portion 14′. When the hydrator assembly 150 is not being used or is disposed in detached relation to the body portion 14′, a plug or like closure element (not shown for purposes of clarity) may be used to close the opening 160 in the body portion 14′.

The hydrator assembly 150 of the present invention also preferably includes a valve assembly 162 including an externally accessible knob 164 which may be selectively positioned between an opened and a closed position by the hand of the user or patient. When in the open position, as shown in FIG. 14, the valve structure 162 includes an integrally, interiorly formed passage 166 which is disposed to establish fluid communication between the interior 153 of the hydrator body 152 and the opening 155 in the upper end thereof. This of course establishes direct fluid communication with the hollow interior 16 of the body portion 14′ and also with the nasal cavity and nasopharynx once the body portion 14′ is properly applied to and sealed about the nose and nostrils of the user as best shown in FIG. 5.

In operation, prior to fitting the hydrator body 152 to the body portion 14′, it is filled with hot water or saline solution as at 170, while the valve structure 162 is in its opened position. Once filled, the valve structure is moved to its closed position such that solid portions as at 169 of the valve structure 162 serve to block communication between the interior 153 and the opening 155. With the valve in its closed position, the opening 155 is placed in direct communication with the hollow interior 16 of the body portion 14′ by the insertion of the connecting collar 156 through the opening 160 and by establishing sealing engagement with the exterior of the body portion 14′ due to the provision of the aforementioned gasket or seal structure 158. The pressure regulating assembly is then positioned into its collapsed position as shown in FIG. 14. Once in the collapsed position, the valve structure 162 is opened to the position shown in FIG. 14 and water vapor or steam is allowed to pass upwardly and into the hollow interior 16 of the body portion 14′. When adequate nasal cavity and/or nasopharynx pre-hydration has been achieved, the valve structure 162 is again disposed in its closed position. Following pre-hydration, the patient or user swallows and the bellows 33′ is released to assume its expanded position and thereby create an initial negative pressure within the nasal cavity and nasopharynx.

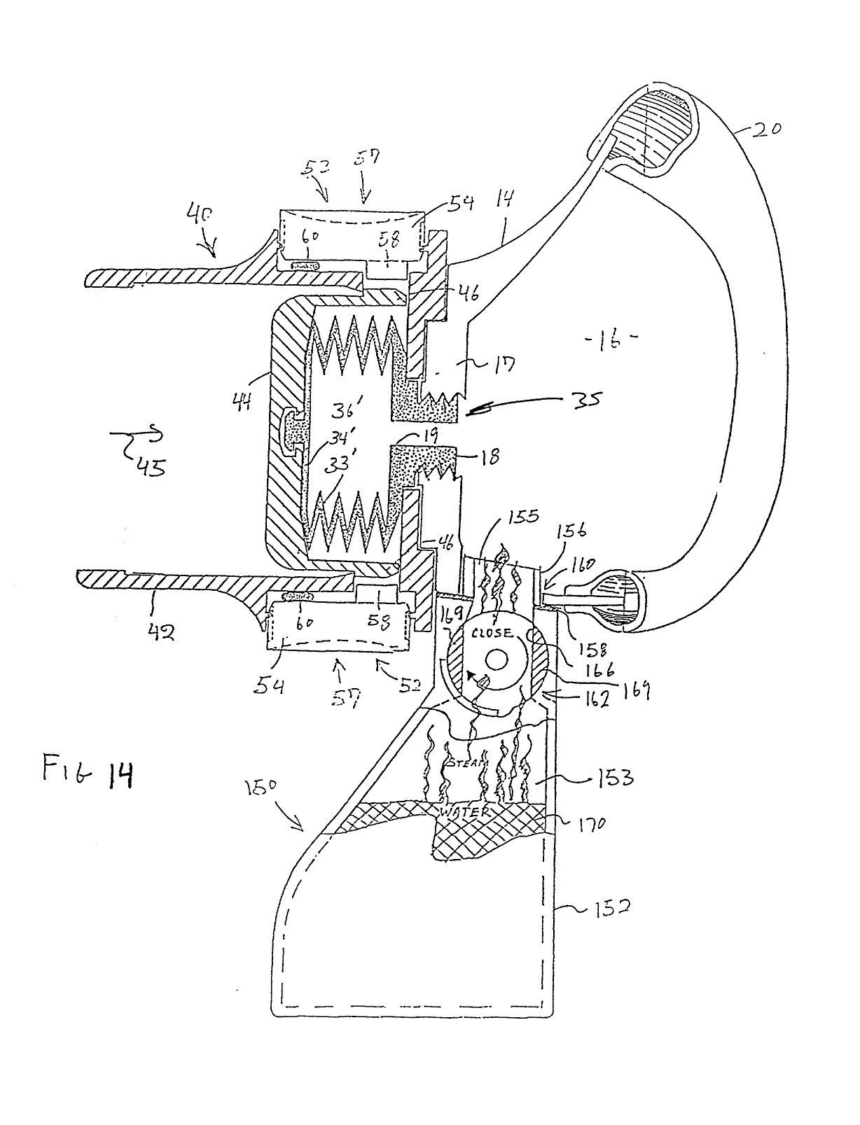

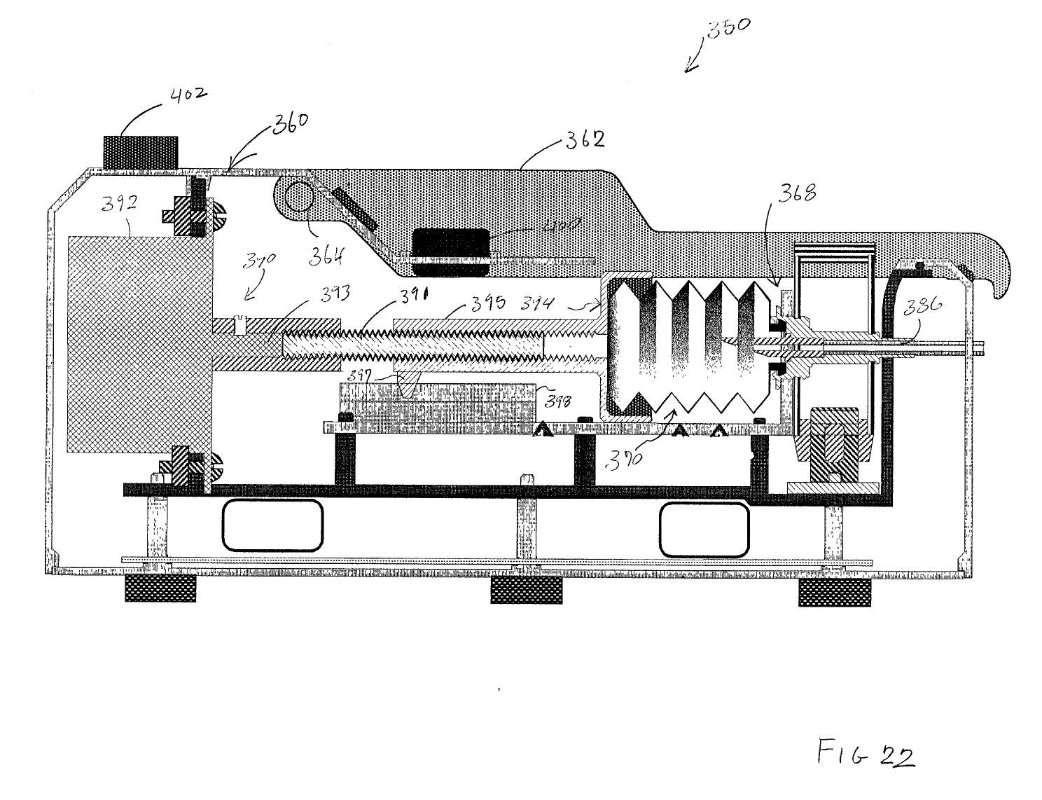

The non-invasive sinus pain reliever of the present invention further comprises an additional preferred embodiment which differs from the other embodiments described herein primarily due to the fact that it is electrically powered rather than being manually operable. More specifically, and with primary reference to FIGS. 15 through 21 this preferred embodiment of the non-invasive sinus pain relieving assembly of the present invention is generally indicated 250. The pain relieving assembly 250 includes an outer casing or housing 252 having an at least partially hollow interior and including a plurality of operative components, including a mask generally indicated as 254 mounted on exterior of the casing 252 and extending outwardly therefrom so as to accomplish the aforementioned sealing engagement with face of the user 200. The mask 254 includes a peripheral seal 255, preferably extending continuously about an open outer end 256 of the body 258 of the mask 254. The body 258 may be formed from a variety of plastic material and include a sufficient structural integrity or rigidity to withstand any stress or forces placed thereon during the presence of a negative pressure within the hollow interior 260 of the mask 254. Further, as with the other embodiments of the subject invention, the dimension and configuration of the mask 254, including the peripheral seal 255, is such as to surround and isolate the nose 210 and nostrils 220 of the user 200 from ambient pressure existing on the exterior of the pain relieving assembly 250. In addition, the sealing engagement between the seal 255 and the face of the user 200 serves to isolate and/or segregate the nose 210 and nostrils 220 from the mouth of the user, thereby isolating exterior communication between the oral and nasal cavities of the user 200. For purposes of clarity, the establishment of the sealing engagement between the peripheral seal structure 255 and the face of the user 200 is specifically disposed in surrounding relation to the nose 210 and nostrils 220 as best demonstrated in FIG. 1, while it is recognized that the embodiment disclosed therein differs significantly from the embodiment of FIGS. 15 through 21.

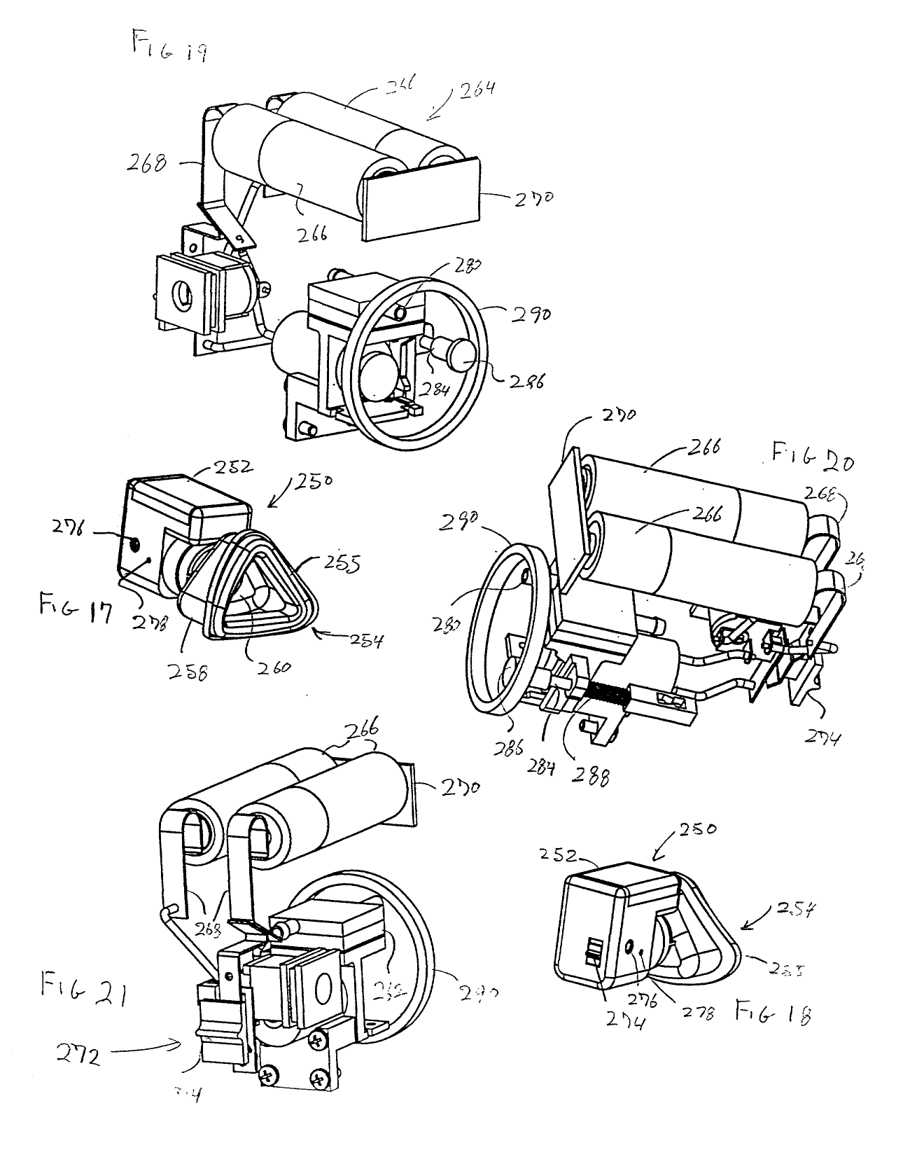

As best shown in FIGS. 19 through 21, the pain relieving assembly 250 comprises a pressure regulator assembly defined by a vacuum pump 262 which is electrically operated and which may be electrically connected to a self contained electrical energy source generally indicated as 264. The energy source 264 may be in the form of one or more removable and/or rechargeable batteries 266. Further, the batteries 266 are electrically connected to the vacuum pump 262 by means of a conductive material contacts 268 and the provision of a conductive material contact plate 270 used to complete the current delivering circuit, including the batteries 266.

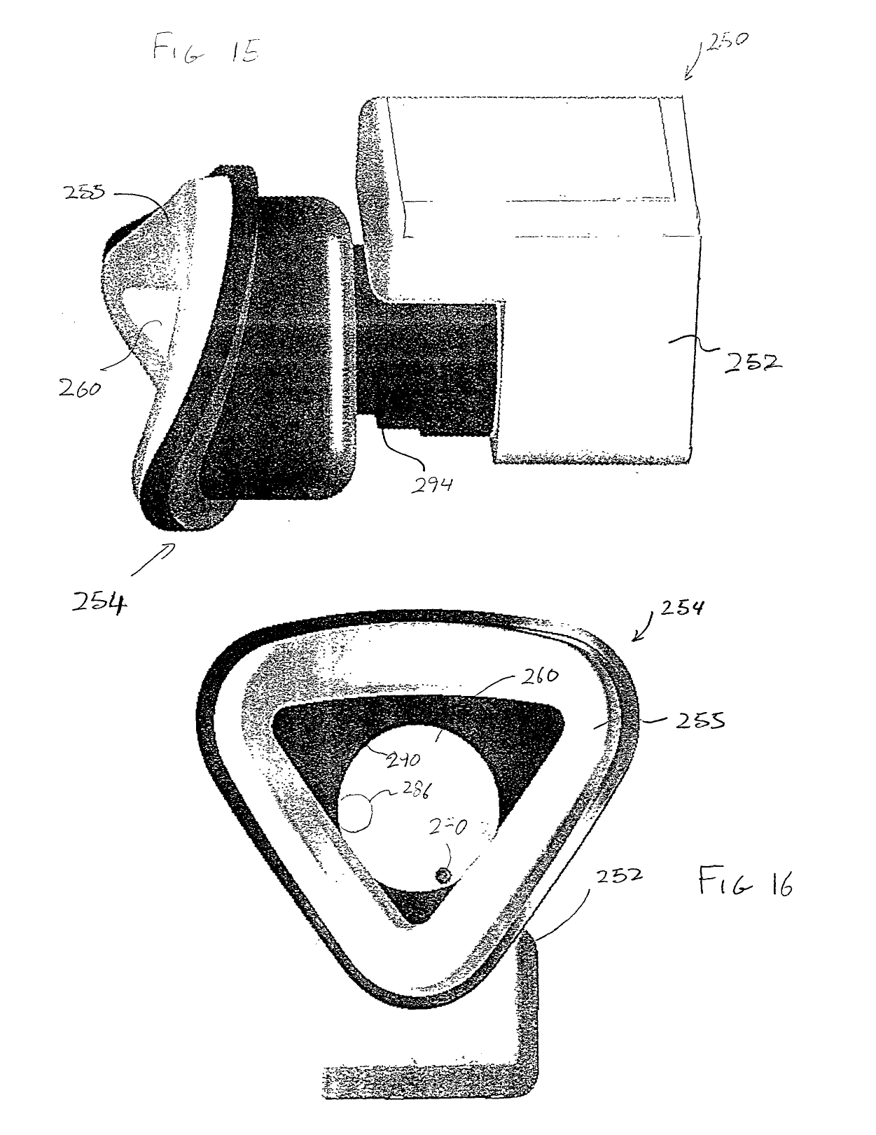

A switch assembly is generally indicated as 272 and includes an on-off switch 274 being exteriorly accessible through an appropriate aperture or port within the casing 252 as, shown in FIGS. 18 and 21. In addition, the casing 252 may include a port 276 disposed in an appropriate location and interconnected to the vacuum pump 262 and/or the circuitry associated with providing electrical energy to the vacuum pump 262 and/or the batteries 266. More specifically, the input port 276 may be utilized to charge the batteries 266 or alternatively to supply an external electrical power source to the vacuum pump 262. Additional features of the casing 262 include a vacuum exhaust or vent port 278, utilized to exhaust air from the interior of the casing during and/or subsequent to the operation of the vacuum pump 262. As shown in FIGS. 15 through 21, the vacuum pump 262 is disposed in direct fluid communication with the interior 260 of the mask 252 by means of a connecting outlet or vacuum port 280. Activation and/or operation of the vacuum pump 262 thereby draws air from the interior 260 of the mask 254 and creates a predetermined negative pressure once the peripheral seal structure 255 is disposed in an operative position on the user's face to establish the aforementioned sealing engagement.

As will be explained in greater detail hereinafter, the vacuum pump 262 is pre-set or programed to draw a specific level of negative pressure or vacuum, which is communicated directly to the interior 260 of the mask 252 once the seal 255 is in the aforementioned sealing engagement with the face of the user 200. In order to accomplish evacuation of any excess fluid, including mucus, air, liquids, etc. from the sinus cavity, a sufficient negative pressure must be transferred to the nasal cavity and nasopharynx. Once a sufficient negative pressure has been established therein, a flow of such excess fluid, from the sinus cavities to the nasal cavity and nasopharynx, occurs. Therefore regulation of the negative pressure within both the interior 260 of the mask 254 as well as the nasal cavity and nasopharynx is necessary and important to accomplish quick pain relieve.

Accordingly, the pain relieving assembly 250 of the embodiment of FIGS. 15 through 21 further includes a regulating assembly which is generally defined by a pressure sensitive member 284. The pressure sensitive member 284 comprises an elongated plunger, rod, or like structure having a distal end 286 disposed in confronting or at least partially movable engagement with a connecting seal 290. The connecting seal 290 is preferably in the form of an O-ring or like seal, gasket or sealing member. The connecting seal 290 facilitates the establishment of a sealing attachment of the inner end 294 (see FIG. 15) to the casing in a manner which prevents access or fluid communication between the exterior of the casing and the interior 260 of the mask 254. Naturally in order to maintain the desired and predetermined negative pressure within the mask, and accordingly within the nasal cavity and nasopharynx, fluid communication between the interior 260 of the mask 252 and the exterior of the mask and/or casing must normally be prevented. However, the pressure relief member 284 is movably mounted on the casing 250 and is disposed in fluid communication with the interior 260 of the mask 254. Because of its position relative to the interior 260 of the mask 254, any exposed surface thereof, such as the enlarged head of the distal end 286, which is exposed to the negative pressure within the interior 260 of the mask 252, will be subjected to forces by the negative pressure. As such, the pressure responsive member 284 is movable between what may be referred to as a “non-venting” position and a “venting” position.

More specifically, the pressure sensitive member 284 includes a biasing spring or other biasing structure 288 mounted thereon or connected thereto. The biasing structure 288 is disposed to normally maintain the pressure sensitive member 284 in the aforementioned non-venting position. However, when the negative pressure or vacuum within the interior of the mask 252 reaches or exceeds a predetermined level or degree, the forces created thereby will react on the distal end 286, so as to longitudinally move or extend the pressure regulating member 284 outwardly against the force of the biasing structure 288. The distal end 286 or other portion of the pressure sensitive member 284 will therefore contact and movably engage the connecting seal 290 causing it to disrupt the sealing attachment of the inner end 294 of the mask 252 with the casing. This displacement of the sealing attachment between the inner end 294 of the mask 252, or any other applicable portion thereof, will cause the establishment of fluid communication between the interior of the mask 260 and the exterior of the mask or casing along some portion of the sealing attachment extending between the mask 254 and the casing 252. Such sealing attachment is defined or facilitated by a normal orientation and placement of the connecting seal 290. However, if and when the negative pressure is again brought within an acceptable, predetermined range or limits, the pressure sensitive member 284 will be directed inwardly because of the influence thereon by the biasing structure 288. The connecting seal 290 will be allowed to assume its normal orientation and again facilitate or define the aforementioned sealing attachment with the casing. Fluid communication between the exterior and interior of the mask 254 will again be prevented.

In use, the user will activate the pressure regulating assembly in the form of the vacuum pump 262 causing negative pressure to be developed within the interior 260 of the mask 254. Activation of the vacuum pump 262 by means of manually operating the switch structure 274 will occur once the aforementioned sealing attachment between the peripheral seal structure 255 and the face of the user has been established. Concurrently, the user will establish the palate lock initially obtained by the user beginning to swallow. This action causes the rearward rotation (in accordance with the direction arrow 24 FIG. 1) of the end of the soft palate or uvula 25 into sealing engagement with the nasopharynx 27. Accordingly, once the sealing engagement between the peripheral seal 255 and the user's face has been established, and the palate lock has been instigated by the user, the nasal cavity and nasopharynx are isolated from the esophagus and exterior ambient pressure. Concurrently, the sinus cavities are brought into direct communication with the nasal cavity and nasopharynx by virtue of connecting cavities and apertures existing in the human body. Therefore as the negative pressure or vacuum develops within the interior 260 of the mask 254 a flow of air will be caused to travel from the interior of the nasal cavity into the hollow interior 260 of the mask 254. This will initially cause a negative pressure within the nasal cavity 28 and nasopharynx 27. A flow of fluid (mucus, liquid, air) will therefore develop from the high pressure zones of the sinus cavities 29 and 30 into the now low pressure zone of the nasal cavity 28 through the aforementioned interconnecting passages and apertures. Once this flow of fluid is developed, the pressure will thereby be equalized between the nasal cavity 28 and the sinus cavities 29 and 30. As set forth above, for purposes of clarity of disclosure, certain linear cross hatching existing in FIG. 1 represents the initial negative pressure being exerted on the nasal cavity 28 and effectively on the sinus cavities 29 an 30 as the negative pressure is created and maintained due to the activation and operation of the electrically powered vacuum pump 262. After use by an individual 200 the assembly 250 can be removed from its operative position by manual operation of the switch structure 274 and by the user 200 “releasing” the palate lock, as by simply completing the swallowing procedure.

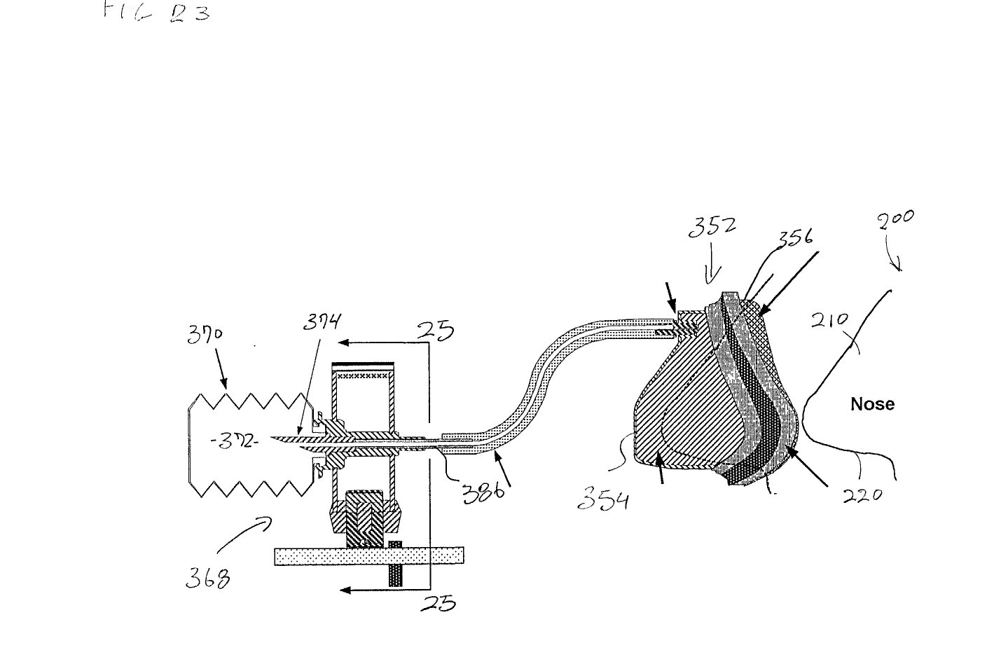

In addition to the above, the sinus pain relieving assembly of the present invention comprises yet another preferred embodiment disclosed in detail in FIGS. 22 through 27. It is emphasized that for purposes of clarity reference will also be made to FIG. 1, wherein the user 200 nose 210 and nostrils 220 are delineated along with the sinus cavity 29 and 30 and nasal cavity 28. In addition, the treatment utilizing this preferred embodiment, as well as an attendant method associated with the assembly generally indicated 350, includes the user creating a “palate lock” in order to accomplish an efficient isolation of the nasal cavity and nasopharynx relative to the esophagus and exterior ambient pressure. As emphasized above, effective isolation of the nasal cavity and nasopharynx facilitates the maintenance of a negative pressure within the nasal cavity to a sufficient degree to accomplish the equalization of the pressures between the sinus cavities 29 and 30 and the nasal cavity 28.

Again, with reference to FIG. 1 the establishment of the aforementioned “palate lock” is indicated by the directional arrow 24 which indicates a rearward rotation of the uvula 25, located at the end of the soft palate, into engaging and substantially sealing relation with the back of the nasopharynx, generally indicated as 27. As emphasized above the establishing of the palate lock facilitates the effective isolation of the nasal cavity and the equalization of pressures between the sinus cavities 29 and 30 and the nasal cavity 28. Further, exterior ambient pressure is prevented from communicating with the nasal cavity 28 by virtue of the fact that the mouth of the user will be closed and the nose 210 and nostrils 220 are maintained in sealed isolation by the operative positioning of the mask 352 associated with the pain relieving assembly 350, as set forth in greater detailed hereinafter.

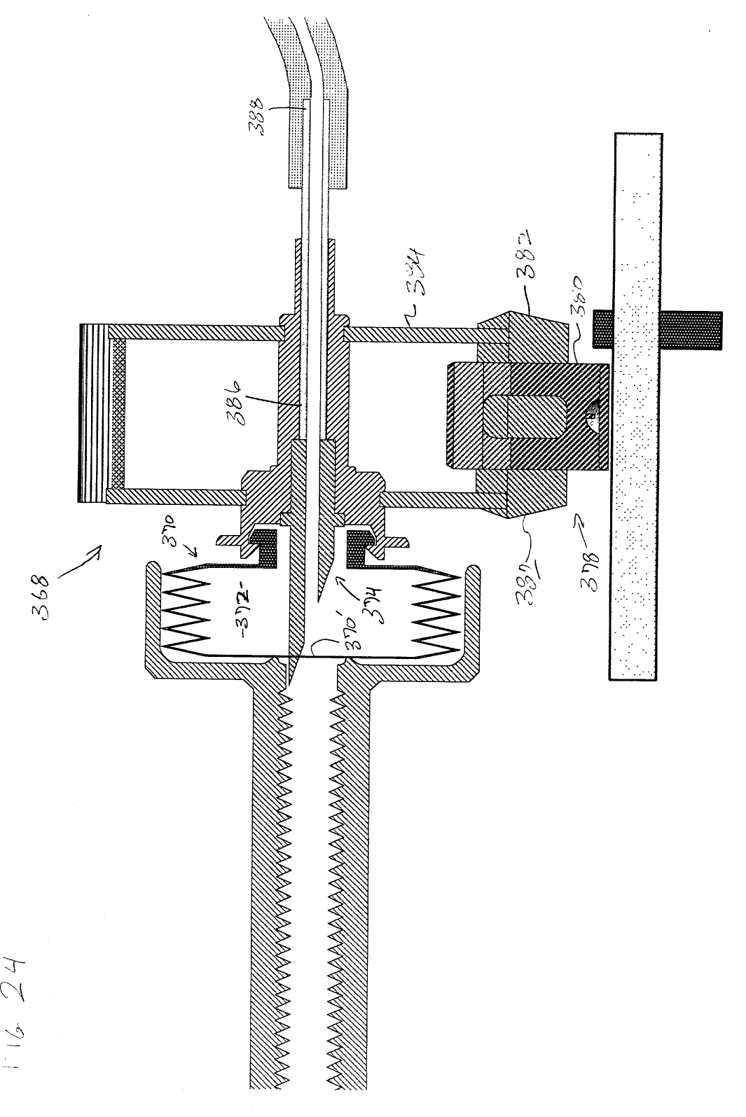

The mask 352 as shown in FIG. 23 includes a body portion 354 and a seal structure 356 which is secured to and extends about the periphery of the open face of the mask. As previously described in the discussion of at least some of the other embodiments of the present invention, the peripheral seal 356 may be at least partially formed from a readily conformable material, such as a hydro-plastic gel or other self conforming material.

The pain relieving assembly 350 of the embodiment of FIGS. 22 through 27 further comprises a housing generally indicated as 360 having an at least partially hollow interior for the mounting and containment of the various operative components of the pain relieving assembly 350. The housing 360 includes a cover, closure or like movably connected portion 362 hingedly of otherwise secured so as to be selectively positionable between an open and a closed position relative to an access opening 366. The access opening 366 is disposed and dimensioned to allow access to and/or removal, replacement, etc. of certain ones of the components, as will be explained in greater detail hereinafter.

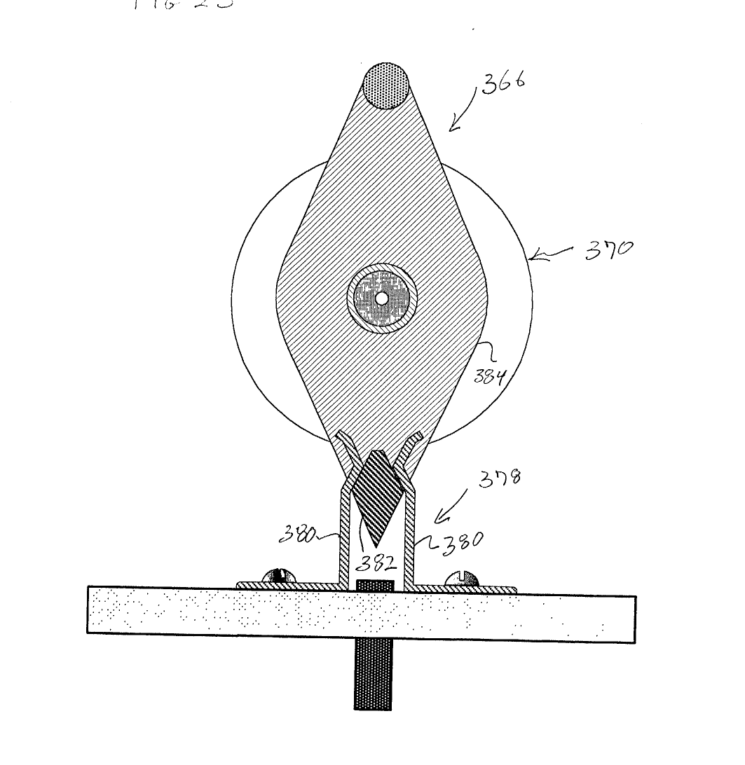

The pain relieving assembly 350 of the present invention further includes a pressure regulating assembly generally indicated as 368. The pressure regulating assembly 368 includes the vacuum chamber 370, which may be defined by a bellows or any other equivalently structured device having a hollow interior 372 and an outer wall formed of a sufficiently flexible and/or resilient material to allow the vacuum chamber 370 to be positioned in either a collapsed position, shown in FIG. 24, or an expanded position shown in FIGS. 22 and 23. As with the vacuum chambers of the previously described embodiments, the disposition of the vacuum chamber 370 in the collapsed position reduces or substantially voids the interior 372 of air. To the contrary, positioning of the vacuum chamber 370 into the expanded position increases the volume of air within the interior 372.

Another structural feature of the pressure regulating assembly 370 is the provision of a safety member generally indicated as 374 and preferably being in the form of a lance or other cutting or penetrating instrument. The safety member 374 is disposed such that when the vacuum chamber 370 is forced into what may be referred to as an “extreme” collapsed position, as shown in FIG. 24, it would be “structurally modified”, such as being cut or penetrated, in a manner which will render it inoperable for further use.

More specifically, in this preferred embodiment, the pain relieving assembly 350, contemplates the removable mounting or attachment of the pressure regulating assembly 368 into an operative position on or within the casing 360 as shown in FIG. 22. Therefore the pressure regulating assembly 368 may define one of a plurality of removably attached and replaceable pressure regulating assemblies, wherein each pressure regulating assembly 368 defines a replaceable unit. It is therefore intended that in certain applications of the pain relieving assembly 350, a new, unused, sterile pressure regulating assembly 368 will be provided for each user or patient.

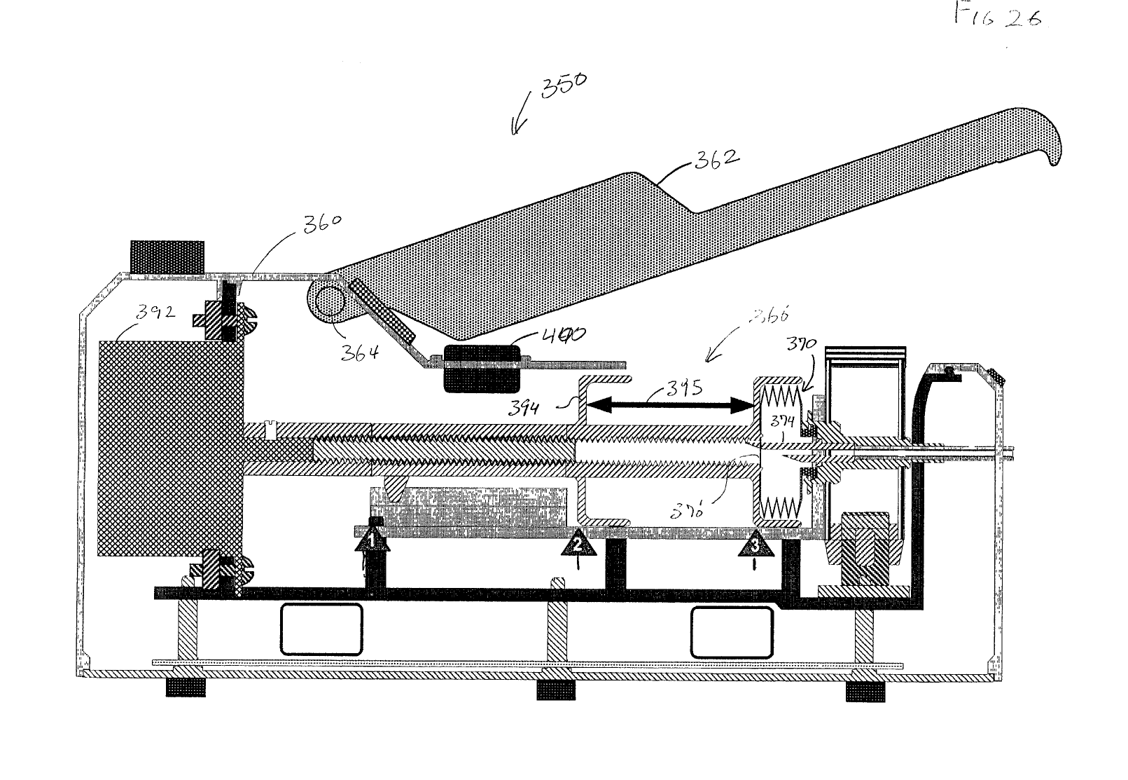

Removable attachment of the pressure regulating assembly 368 includes a mounting assembly generally indicated as 378 and shown in detail in FIGS. 24 and 25. The mounting assembly 378 may include a bracket, clip or like structure having two upwardly extending spaced apart gripping arms 380 collectively disposed and structured to removably receive and be attached to a connector or attachment member 382 fixedly secured to a base 384 of the pressure regulating assembly 368. Further structural details of the pressure regulating assembly 368 include a centrally disposed tube or conduit 386 having a free outer end 388 and being secured in direct fluid communication with the safety member 374. Accordingly, the pressure regulating assembly 368 of the pain relieving assembly 350 includes the attached vacuum chamber 370, safety member 374 base 384 and attachment or connector member 382 as a single replaceable unit intended to be removed from the casing 360 after a single use. In order to assure only a single use of each of the replaceable pressure regulating assemblies 368, the vacuum chambers associated therewith can be structurally modified, by being pierced or cut by the safety member 374, thereby making them incapable of reuse. Penetration of each of the vacuum chambers 370, by the safety member 374, occurs when the respective vacuum chambers are disposed in the extreme collapsed position as shown in FIG. 24.

Another feature of the pain relieving assembly 360 of the present invention is a provision of a positioning assembly generally indicated as 390 mounted on or within the casing 360. The positioning assembly 390 includes a drive structure 392 and a positioning member 394. The positioning member 394 may be in the form of a carriage or other structure disposed in an orienting engagement with the vacuum chamber 370. Further, the positioning member 394 is connected in driven relation to the drive structure 392. The drive structure 392 is preferably in the form of a drive motor which may or may not be electrically powered, but is specifically structured to automatically drive or dispose the positioning member 394 in a manner which accomplishes the intended positioning of the vacuum chamber 370 into and out of both the collapsed position and the expanded position, as described above.

Therefore, while the most preferred embodiment of the paining relieving assembly 350 comprises the drive structure 392 being in the form of an electrically powered, reversible rotary drive motor, other substantially equivalent drive structures could be utilized which are specifically operable to “automatically” position the vacuum chamber 370 into and between each of the collapsed and expanded positions without manual positioning being applied to the vacuum chamber 370 as is required in certain of the other, above noted embodiments of the present invention. Such additional equivalent structures which may define the automatic drive structure 392 could include a spring motor, biasing structure or other like device.

In the preferred embodiment, the drive structure 392 comprises the aforementioned reversible drive motor which rotates a drive shaft 393. The drive shaft 393 is drivingly linked or connected to a secondary shaft 391 which is externally threaded so as to matingly engage the interiorly threaded configuration of an adapter portion 395 of the positioning member or carriage 394. Accordingly, activation of the drive motor or drive structure 392 causes rotation of drive shaft 393 and secured secondary shaft 391. As a result, the elongated internally threaded adaptor 395 moves axially along the length of the secondary shaft 391. A restrictor device 397 is fixedly secured to the adaptor 395 and slides or moves longitudinally with the adaptor 395, relative to a stop member 398. However engagement between the restrictor member 397 and the stop member 398 prevents rotation of the adaptor 395 and affects axial movement thereof as well as the carriage portion 394 secured to the adaptor 395. The vacuum chamber 370 is thereby oriented between the expanded and collapsed positions, dependent on the direction of rotation of the drive motor 392.

The preferred embodiment of the pain relieving assembly 350 also includes a control assembly which may comprise appropriate and somewhat conventional electronic circuitry and/or interconnections between a power supply, which energizes the drive motor 392 and at least one but preferably a plurality of activating control members or buttons. More specifically the control assembly includes an activating load button 400 and an activating treatment button 402. The control assembly (not shown for purposes of clarity) is cooperatively structured, with the structural and operative features of the drive motor 392 such that the drive motor 392 may activate the pressure regulating assembly 368 through what may be referred to as a “load cycle” and a “treatment cycle”.

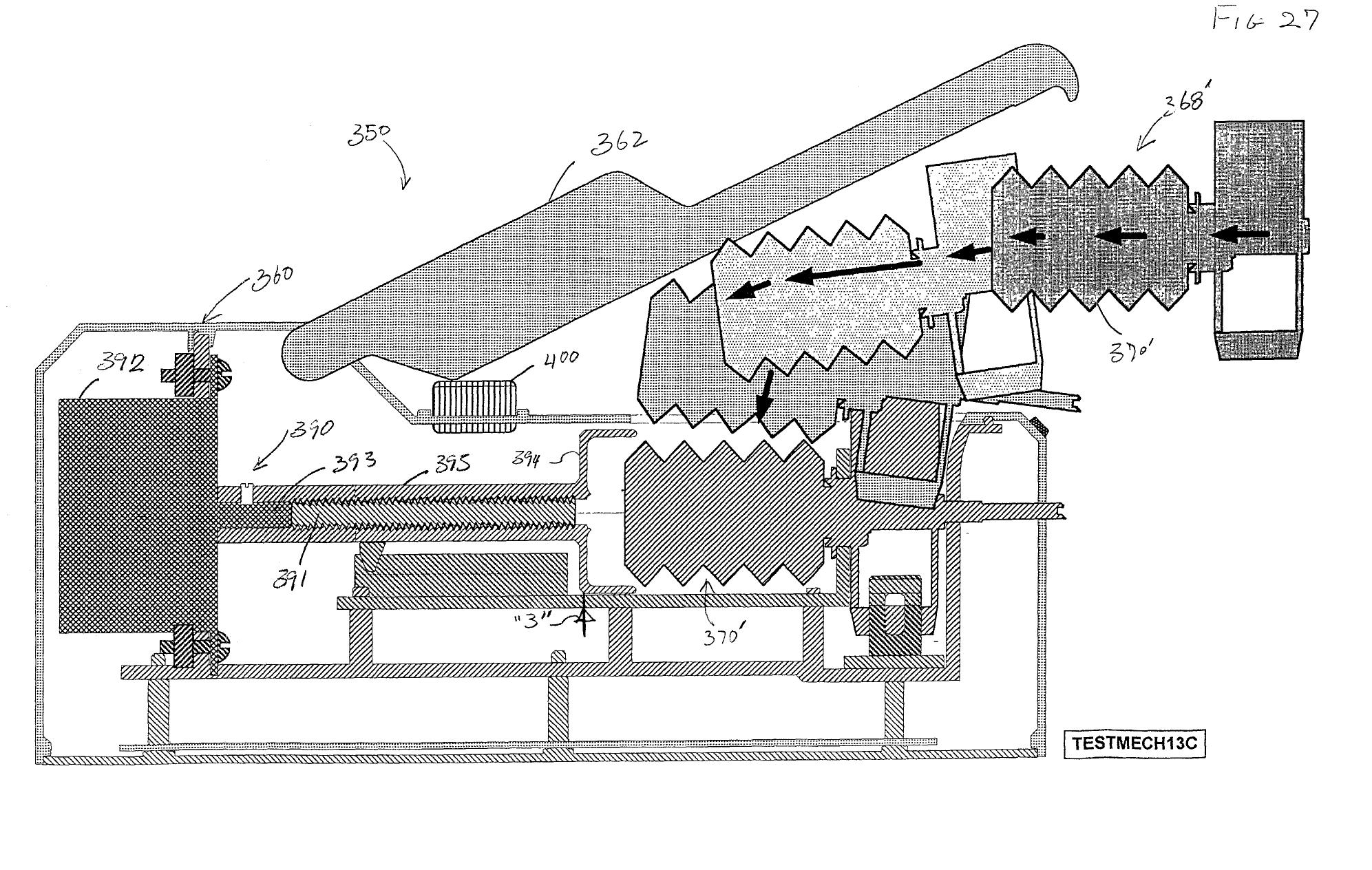

With primary reference 22 and 26, the vacuum chamber 370, after being used, is positioned in the orientation primarily shown in FIG. 22. As such the driving member or carriage 394 is disposed in the original orienting engagement with the vacuum chamber 370. In this position it would be difficult or impossible to remove the pressure regulating assembly 368 from its intended position on or within the casing 360, even though it has already been used. Accordingly, removal is accomplished by lifting the cover 362 so as to provide access to the interior thereof through the access opening 366. Once cover 362 is opened, as shown in FIG. 26, access to the load button or control member 400 is available to the user or medical personnel operating the pain relieving assembly 350.

Upon depression of the load button or control member 400 the control assembly activates the drive structure 392 so as to operate in the aforementioned load cycle. The load cycle serves to orient the vacuum chamber 370 into the aforementioned “extreme” collapsed position such that its outer most end 370′ is forced into an almost completely or totally collapsed position indicated by the position arrow “3” in FIGS. 22 and 26. In such position safety member or lance 374 cuts or penetrates a portion of the wall as at 370′ (see FIGS. 24 and 26) of the vacuum chamber 370 thereby structurally modifying the vacuum chamber 370 so as to render it inoperative for future use or reuse. Once so modified, the vacuum chamber 370 is allowed to assume an expanded position shown in FIG. 22. In addition, the carriage or positioning 394 extends beyond the normally expanded position into a spaced apart position relative to the vacuum chamber 370, as indicated by directional arrow “2” in FIG. 27. Directional arrow 395 serves to indicate the degree of travel of carriage 394 between the extreme collapsed position of position arrow “3” and the completely expanded and detached position indicated by position arrow “2”. When the carriage 394 is disposed in the position indicated by positional arrow “2” in FIG. 27 the previously used vacuum chamber 370 and pressure regulating assembly 368 may be removed and replaced by a new, unused and preferably sterile pressure regulating assembly 368′ including the unused chamber 370′.

Since many modifications, variations and changes in detail, such as the use of a small vacuum pump, can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A non-invasive sinus pain relieving assembly comprising:
a) a housing including a pressure regulating assembly connected thereto,
b) a mask connected in fluid communication with said pressure regulating assembly and including a peripheral seal,
c) said seal dimensioned and configured to define a sealing engagement with the face of a user; said sealing engagement disposed in isolating relation to the nose and nostrils relative to ambient pressure and to the mouth of the user,
d) a positioning assembly mounted on said housing in orienting engagement with said pressure regulating assembly, and
e) said positioning assembly operable upon establishment of said sealing engagement to operatively position said pressure regulating assembly and creating negative pressure within the nasal cavity and nasopharynx sufficient to define a fluid transfer from the sinus cavities to the nasal cavity, upon the user creating a palate lock.

2. An assembly as recited in claim 1 wherein said positioning assembly comprises an automatic drive structure and a positioning member connected in driving relation to said automatic drive structure and in driving relation to said pressure regulating assembly.

3. An assembly as recited in claim 2 wherein said auto drive structure comprises an electrically power motor.

4. An assembly as recited in claim 2 wherein said automatic drive structure comprises a rotary, reversible electric power driving motor.

5. An assembly as recited in claim 2 wherein said positioning member is disposed in orienting relation to said pressure regulating assembly.

6. An assembly as recited in claim 5 wherein said pressure regulating assembly comprises a vacuum chamber disposable into both an expanded position and a collapsed position upon activation of said drive motor.

7. An assembly as recited in claim 6 wherein said collapsed position is defined by a reduced volume of air within said vacuum chamber and said expanded position being defined by an increased volume of air within said vacuum chamber; said negative pressure generated by said vacuum chamber passing from said collapsed position into said expanded position.

8. An assembly as recited in claim 6 wherein said positioning member is disposed in driving engagement with said vacuum chamber, said vacuum chamber movable from said expanded position to said collapsed position and from said collapsed position to said expanded position upon reversed operation of said drive structure in a first and second direction respectively.

9. An assembly as recited in claim 1 wherein said positioning assembly comprises a carriage movably mounting within said housing and connected in driving relation to said pressure regulating assembly.

10. An assembly as recited in claim 9 wherein said pressure regulating assembly comprises a vacuum chamber disposable out of both a collapsed position and an expanded position, said carriage movable with said vacuum chamber between said collapsed and expanded positions.

11. An assembly as recited in claim 10 wherein said positioning assembly further comprises a drive motor movably connected in driving relation to said carriage, said drive motor operable through a treatment cycle so as to create said negative pressure within said mask.

12. An assembly as recited in claim 11 wherein said drive motor is reversible and electrically powered.

13. An assembly as recited in claim 11 wherein said pressure regulating assembly comprises one of a plurality of replaceable units removably mounted within said housing in said orienting engagement with said positioning assembly and in fluid communication with said mask.

14. An assembly as recited in claim 13 wherein said drive motor is operable through a load cycle structure to prevent re-use of anyone of said pressure regulating assembly defining one of said plurality of replaceable units.

15. An assembly as recited in claim 14 wherein said pressure regulating assembly further comprises a safety member disposed in engageable relation to said vacuum chamber during said load cycle, said safety member structured to an operatively modify said vacuum chamber upon predetermined engagement therewith.

16. An assembly as recited in claim 15 wherein said predetermine engagement is define by penetrating orientation of said safety member relative to said vacuum chamber.

17. An assembly as recited in claim 1 wherein said pressure regulating assembly comprises one of a plurality of replaceable units removably mounted within said housing in said orienting engagement with said positioning assembly and in fluid communication with said mask.

18. An assembly as recited in claim 17 wherein at least some of said plurality of units comprise a pressure regulating assembly including a vacuum chamber and safety member, said vacuum chamber positionable into and out of both a collapsed position and an expanded position and said safety member structure to in-operatively modifies said vacuum chamber upon predetermine engagement therewith.

19. An assembly as recited in claim 18 wherein said predetermine engagement is defined by penetrating orientation of said safety member relative to said vacuum chamber.

20. An assembly as recited in claim 18 wherein said mask is removably connected in fluid communication to said vacuum chamber.

21. An assembly as recited in claim 18 wherein said positioning assembly comprises a drive motor and a carriage connected in driving relation to said drive motor, said carriage connected in driving relation to said vacuum chamber for the positioning thereof into and out of said collapsed for expanded positions.

22. An assembly as recited in claim 21 wherein said drive motor is operable through a treatment cycle so as to create said negative pressure within said mask.

23. An assembly as recited in claim 22 wherein said drive motor is operable through a load cycle structure to prevent re-use of said pressure regulating assembly defining anyone of said plurality of replaceable units.

24. An assembly as recited in claim 23 wherein said pressure regulating assembly further comprises a safety member disposed in engagable relation to said vacuum chamber during said load cycle, said safety member structure to in-operatively modifies said vacuum chamber upon predetermined engagement therewith.

25. An assembly as recited in claim 24 wherein said predetermine engagement is defined by penetrating orientation of said safety member relative to said vacuum chamber.

26. A non-invasive sinus pain relieving assembly comprising:
a) a housing including a pressure regulating assembly mounted thereon,
b) a mask connected in fluid communication with said pressure regulating assembly and including a peripheral seal dimensioned and configured to establish a sealing engagement with the face of a user,
c) a positioning assembly mounted on said housing in orienting engagement with said pressure regulating assembly,
d) a control assembly operatively connected to said positioning assembly and structured to actuate positioning of said pressure regulating assembly through a load cycle and a treatment cycle, and
e) said pressure regulating assembly operable to generate a pre-determine negative pressure transferable to said mask and the nasal cavity of the user at least when said pressure regulating assembly is operative in said treatment cycle.

27. An assembly as recited in claim 26 wherein said pressure regulating assembly comprises one of a plurality of replaceable units removably mounted on said casing in said orienting engagement with said positioning assembly.

28. An assembly as recited in claim 27 wherein said cycle is at least partially define by said pressure regulating assembly mounted on said housing being inoperatively modified to prevent use thereof.

29. An assembly as recited in claim 28 wherein said pressure regulating assembly comprises a vacuum chamber structured to generate a negative pressure during said treatment cycle and a safety member disposed in engagable relation to said vacuum chamber during said load cycle, said safety member structure to and operatively modified said vacuum chamber upon predetermine engagement therewith.

30. An assembly as recited in claim 26 wherein said pressure regulating assembly comprises a vacuum chamber; said treatment cycle at least partially defined by activation of said positioning assembly into driving engagement with said vacuum chamber and sequential orientation thereof into a collapsed position and an expanded position.

31. An assembly as recited in claim 30 wherein said mask and said vacuum chamber are cooperatively structure to transfer said negative pressure created by said vacuum chamber, when being oriented in said expanded position, to said mask and the nasal cavity and nasopharynx of the user upon the user establishing a palate lock.

32. An assembly as recited in claim 31 wherein said pressure regulating assembly comprises one of a plurality of replaceable units removably mounting on said casing in said orienting engagement with said positioning assembly.

33. An assembly as recited in claim 32 wherein said load cycle is at least partially define by said pressure regulating assembly mounted on said housing being inoperatively modified to prevent use thereof.

34. An assembly as recited in claim 33 wherein said pressure regulating assembly further comprises a safety member disposed in engageable relation to said vacuum chamber during said load cycle, said safety member structure to inoperatively modified said vacuum chamber upon predetermine engagement therewith.

35. An assembly as recited in claim 26 wherein said positioning assembly comprises an automatic drive structure and a positioning member connected in driving relation to said automatic drive structure and in driving relation to said pressure regulating assembly, said positioning member being disposed in orienting relation to said pressure regulating assembly.

36. An assembly as recited in claim 26 wherein said mask is movably connected and selectively positionable into said sealing engagement on the user's face at a spaced apart location from said housing.

37. An assembly as recited in claim 36 further comprising an elongated conduit interconnected between said pressure regulating assembly and said mask and structure to establish fluid communication therebetween.

38. An assembly as recited in claim 37 wherein said pressure regulating assembly comprises one of a plurality of replaceable units removably mounted on said casing in said orienting engagement with said positioning assembly; said conduit removably attachable to anyone of said replaceable units defining said pressure regulating assembly and mounted on said housing.
